(19) United States
(12) Patent Application Publication
(10) Pub. No.: US 2024/0229141 A1
(43) Pub. Date: Jul. 11, 2024

(54) METHODS AND COMPOSITIONS FOR CLASSIFYING DLBCL

(71) Applicant: Roche Molecular Systems, Inc., Pleasanton, CA (US)

(72) Inventors: Rajiv Dua, Manteca, CA (US); Marcel Fontecha, San Ramon, CA (US); Yan Li, Palo Alto, CA (US); Wei-min Liu, Dublin, CA (US); Christopher Santini, Pleasant Hill, CA (US); Lori Steiner, Alameda, CA (US); Yu Chuan Tai, Pleasanton, CA (US)

(21) Appl. No.: 17/447,978

(22) Filed: Sep. 17, 2021

Related U.S. Application Data
(63) Continuation of application No. 16/098,019, filed on Oct. 31, 2018, now Pat. No. 11,124,840.
(60) Provisional application No. 62/336,486, filed on May 13, 2016.

(51) Int. Cl. C12Q 1/6886

(52) U.S. Cl. CPC C12Q 1/6886; C12Q 2600/106; C12Q 2600/112; C12Q 2600/158; C12Q 2600/16

(57) ABSTRACT

Provided herein are methods and compositions to classify DLBCL subtypes using quantitative RT-PCR.

### BACKGROUND OF THE INVENTION

Diffuse Large B-Cell Lymphoma (DLBCL) comprises 30-35% of all Non-Hodgkin lymphoma. DLBCL is biologically aggressive, but can be cured in >50% of the cases. However, up to one third of the patients develop resistance and are refractory to the treatments. The standard treatment is chemotherapy CHOP or chemotherapy+Rituxamab (R-CHOP). DLBCL can be classified into three different molecular cell-of-origin (COO) subtypes: germinal center B-cell (GCB), activated B-cell (ABC), and primary mediastinal B-cell lymphoma (PMBCL). Retrospective analysis by the Lymphoma/Leukemia molecular profiling project demonstrated that DLBCL patients with GCB subtype have better prognosis than those with ABC subtype when treated with R-CHOP, and drug candidates to improve ABC subtype prognosis are in development.

Current methods for distinguishing GCB and ABC subtypes include immunohistochemistry (IHC) and gene expression profiling. IHC and gene expression profiling technologies are time consuming, and have additional drawbacks for subtype classification. For example, gene expression technology uses frozen samples and not the formaldehyde fixed paraffin embedded tissue (FFPET) specimens that are typically collected in clinical laboratories. Nanostring Technologies (Seattle, WA) has developed a gene expression profiling signature that classifies DLBCL subtypes using FFPET samples, but the Nanostring platform is not widely adopted in the marketplace and it is expensive. IHC also uses FFPET samples but shows high assay variability across laboratories.

### SUMMARY OF THE INVENTION

Provided herein are methods and compositions for determining diffuse large B cell lymphoma (DLBCL) subtype and treating DLBCL patients. Provided herein are methods and compositions for determining diffuse large B cell lymphoma (DLBCL) subtype and treating DLBCL patients. In some embodiments, provided are methods of identifying an individual with DLBCL comprising: (a) obtaining a sample from the individual (DLBCL sample); (b) detecting by qRT-PCR the expression of GCB markers ZNF318, PDK3, HMGN1, PTK2, SSBP2, BCL6, and/or LRMP in the DLBCL sample; (c) detecting by qRT-PCR the expression of ABC markers ARID3A, CCND2, FOXP1, KIAA0226L, JADE3, PIM2, TCF4, and/or FAM46C in the DLBCL sample and the control sample; and (d) detecting by qRT-PCR the expression of a control gene (e.g., internal control) in the DLBCL sample; wherein the ratio of GCB marker expression to ABC marker expression being higher than a GCB threshold value in said individual's sample indicates sensitivity of said individual to the administration of R-CHOP (rituximab or etoposide; cyclophosphamide; doxorubicin; vincristine; and prednisolone). Some embodiments, if the ratio of ABC marker expression to GCB marker expression is higher than an ABC threshold value in the individual's sample indicate sensitivity of the individual to an alternative administration. In some embodiments, the method further comprises adjusting the level of expression detected for the genes in steps (b) and (c) based on the expression detected of the control gene in (d). In some embodiments, the administration is provided directly to the patient.

In some embodiments, provided are methods of providing treatment for an individual with DLBCL comprising: (a) obtaining a sample from the individual (DLBCL sample); (b) detecting by qRT-PCR the expression of GCB markers ZNF318, PDK3, HMGN1, PTK2, SSBP2, BCL6, and/or LRMP in the DLBCL sample; (c) detecting by qRT-PCR the expression of ABC markers ARID3A, CCND2, FOXP1, KIAA0226L, JADE3, PIM2, TCF4, and/or FAM46C in the DLBCL sample and the control sample; (d) detecting by qRT-PCR the expression of a control gene (e.g., internal control) in the DLBCL sample; and (e) providing treatment for the individual. In some embodiments, the treatment comprises administration of R-CHOP (rituximab or etoposide; cyclophosphamide; doxorubicin; vincristine; and prednisolone) if the ratio of GCB marker expression to ABC marker expression is higher than a GCB threshold value. In some embodiments, the treatment comprises an alternative therapy if the ratio of ABC marker expression to GCB marker expression is higher than an ABC threshold value. In some embodiments, the method further comprises adjusting the level of expression detected for the genes in steps (b) and (c) based on the expression detected of the control gene in (d). In some embodiments, the treatment is provided directly to the patient.

In some embodiments, 1, 2, 3, 4, 5, or 6 GCB markers are detected in step (b) in any combination. In some embodiments, all 7 GCB markers are detected in step (b). In some embodiments, 1, 2, 3, 4, 5, 6, or 7 ABC markers are detected in step (c) in any combination. In some embodiments, all 8 ABC markers are detected in step (c). In some embodiments, step (b) comprises detecting the expression of ZNF318, SSBP2, and PTK2. In some embodiments, step (c) comprises detecting the expression of CCND2, FOXP1, and JADE3.

In some embodiments, the methods further comprise carrying out steps (b)-(d) on a GCB positive control, and the result used to set the GCB threshold value. In some embodiments, the GCB positive control comprises 51-100% known GCB sample, e.g., 55-85%, 55-65%,

60-70% known GCB sample. In some embodiments, the remaining GCB positive control is comprised of known ABC sample. In some embodiments, the method further comprises carrying out steps (b)-(d) on an ABC positive control, and the result used to set the ABC threshold value. In some embodiments, the ABC positive control comprises 51-100% known ABC sample, e.g., 55-85%, 55-65%, 60-70% known ABC sample. In some embodiments, the remaining ABC positive control is comprised of known GCB sample. In some embodiments, the method further comprises carrying out steps (b)-(d) on a negative control sample, e.g., a sample lacking nucleic acids, a non-cancer sample, or a sample substantially lacking the recited ABC and GCB marker nucleic acids.

In some embodiments, the sample is from lung biopsy (e.g., tumor tissue) or bronchoalveolar lavage. In some embodiments, the sample is formalin-fixed paraffin embedded tissue (FFPET), e.g., from a tumor sample, either in the lung or metastasized. In some embodiments, the sample is blood, plasma, serum, urine, mucous, mucosal tissue, or saliva.

In some embodiments, the detecting of (b) and (c) are carried out in multiplex in multiple vessels. For example, the detecting of (b) can be carried out in 1-6 vessels, with each of the GCB markers detected using a different label for each GCB marker probe, or using the same label on two or more GCB marker probes. Similarly, the detecting of (c) can be carried out in 1-7 vessels, with each of the ABC markers detected using a different label for each ABC marker probe, or using the same label on two or more ABC marker probes. In some embodiments, each GCB and ABC marker is individually detected. In some embodiments, the detecting of (b) is carried out in a single vessel for each sample. In some embodiments, the detecting of (c) is carried out in a single vessel for each sample. In some embodiments, the detecting of (d) is carried out in the same vessel(s) as the detecting of (b) and (c).

In some embodiments, the alternative administration or therapy includes a BTK inhibitor, SYK inhibitor, NFkB inhibitor, or immunomodulatory agent. In some embodiments, the alternative administration or therapy comprises R-CHOP, alone or in combination with a BTK inhibitor, SYK inhibitor, NFkB inhibitor, or immunomodulatory agent.

Further provided are methods for determining the cell of origin (COO) subtype for an individual with DLBCL comprising (a) obtaining a sample from the individual (DLBCL sample); (b) detecting by qRT-PCR the expression of GCB markers ZNF318, PDK3, HMGN1, PTK2, SSBP2, BCL6, and/or LRMP in the DLBCL sample; (c) detecting by qRT-PCR the expression of ABC markers ARID3A, CCND2, FOXP1, KIAA0226L, JADE3, PIM2, TCF4, and/or FAM46C in the DLBCL sample; (d) detecting by qRT-PCR the expression of a control gene in the DLBCL sample; and (e) determining that the COO subtype of the individual is (i) germinal center B cell (GCB) if the ratio of GCB marker expression to ABC marker expression is higher than a GCB threshold value, or (ii) activated B cell (ABC) if the ratio of ABC marker expression to GCB marker expression is higher than an ABC threshold value. In some embodiments, the method further comprises adjusting the level of expression detected for the genes in steps (b) and (c) based on the expression detected of the control gene in (d).

In some embodiments, 1, 2, 3, 4, 5, or 6 GCB markers are detected in step (b) in any combination. In some embodiments, all 7 GCB markers are detected in step (b). In some embodiments, 1, 2, 3, 4, 5, 6, or 7 ABC markers are detected in step (c) in any combination. In some embodiments, all 8 ABC markers are detected in step (c). In some embodiments, step (b) comprises detecting the expression of ZNF318, SSBP2, and PTK2. In some embodiments, step (c) comprises detecting the expression of CCND2, FOXP1, and JADE3.

In some embodiments, the methods further comprise carrying out steps (b)-(d) on a GCB positive control, and the result used to set the GCB threshold value. In some embodiments, the GCB positive control comprises 51-100% known GCB sample, e.g., 55-85%, 55-65%, 60-70% known GCB sample. In some embodiments, the remaining GCB positive control is comprised of known ABC sample. In some embodiments, the method further comprises carrying out steps (b)-(d) on an ABC positive control, and the result used to set the ABC threshold value. In some embodiments, the ABC positive control comprises 51-100% known ABC sample, e.g., 55-85%, 55-65%, 60-70% known ABC sample. In some embodiments, the remaining ABC positive control is comprised of known GCB sample. In some embodiments, the method further comprises carrying out steps (b)-(d) on a negative control sample.

In some embodiments, the sample is from lung biopsy (e.g., tumor tissue) or bronchoalveolar lavage. In some embodiments, the sample is formalin-fixed paraffin embedded tissue (FFPET), e.g., from a tumor sample, either in the lung or metastasized. In some embodiments, the sample is blood, plasma, serum, urine, mucous, mucosal tissue, or saliva.

In some embodiments, the detecting of (b) and (c) are carried out in multiplex in multiple vessels. For example, the detecting of (b) can be carried out in 1-6 vessels, with each of the GCB markers detected using a different label for each GCB marker probe, or using the same label on two or more GCB marker probes. Similarly, the detecting of (c) can be carried out in 1-7 vessels, with each of the ABC markers detected using a different label for each ABC marker probe, or using the same label on two or more ABC marker probes. In some embodiments, each GCB and ABC marker is individually detected. In some embodiments, the detecting of (b) is carried out in a single vessel for each sample. In some embodiments, the detecting of (c) is carried out in a single vessel for each sample. In some embodiments, the detecting of (d) is carried out in the same vessel(s) as the detecting of (b) and (c).

In some embodiments, the method further comprises providing treatment for the individual depending on the COO subtype.

Further provided are kits for determining the COO subtype of an individual with DLBCL. In some embodiments, the kit comprises (a) a mixture comprising a primer set and a fluorescently labeled probe that specifically amplifies and detects at least one of GCB marker ZNF318, PDK3, HMGN1, PTK2, SSBP2, BCL6, and LRMP gene products (e.g., 2, 3, 4, 5, 6, or all 7); and (b) a mixture comprising a primer set and a fluorescently labeled probe that specifically amplifies and detects at least one of ABC marker ARID3A, CCND2, FOXP1, KIAA0226L, JADE3, PIM2, TCF4, and FAM46C gene products (e.g., 2, 3, 4, 5, 6, 7, or all 8). In some embodiments, the kit includes primer sets and probes to specifically amplify and detect all 7 GCB markers and all 8 ABC markers. In some embodiments, mixture (a) comprises a primer set and a fluorescently labeled probe that specifically amplifies and detects ZNF318, SSBP2, and PTK2. In some embodiments, mixture (b) comprises a primer set and a fluorescently labeled probe that specifically amplifies and detects CCND2, FOXP1, and JADE3. In some embodiments, the mixtures of (a) and (b) each further comprise a primer set and a fluorescently labeled probe that specifically amplifies and detects a control gene product, wherein the fluorescently labeled probe that specifically detects the control gene product is differently labeled than the fluorescently labeled probes in mixture (a) and mixture (b). In some embodiments, the fluorescently labeled probes in mixture (a) are all labeled with the same fluorescent label. In some embodiments, the fluorescently labeled probes in mixture (b) are all labeled with the same fluorescent label.

In some embodiments, the kit comprises a plurality of mixtures that comprise a primer set and a fluorescently labeled probe that specifically amplifies and individually detects (i) each of GCB marker ZNF318, PDK3, HMGN1, PTK2, SSBP2, BCL6, and LRMP gene products; (ii) each of ABC marker ARID3A, CCND2, FOXP1, KIAA0226L, JADE3, PIM2, TCF4, and FAM46C gene products; and (iii) a control gene product, wherein the primer set and fluorescently labeled probe that specifically amplifies and individually detects the control gene product are present in each of the plurality of mixtures. In some embodiments, the kit comprises 3-15 mixtures, e.g., 5 mixtures. In some embodiments, the kit comprises a plurality of mixtures that comprise a primer set and a fluorescently labeled probe that specifically amplifies and individually detects (i) each of ZNF318, PDK2, and SSBP2; (ii) each of CCND2, FOXP1, and JADE3; and (iii) a control gene product, wherein the primer set and fluorescently labeled probe that specifically amplifies and individually detects the control gene product are present in each of the plurality of mixtures.

In some embodiments, the kit further comprises reverse transcriptase and/or thermostable DNA polymerase. In some embodiments, the kit further comprises an enzyme with reverse transcriptase and DNA polymerase activity. In some embodiments, the kit further comprises at least one control sample, e.g., an ABC positive control and/or GCB positive control, as described herein. In some embodiments, the kit further includes a negative control (e.g., non-cancer sample).

In some embodiments, the primer set that specifically amplifies ZNF318 is a forward and reverse primer having sequences selected from SEQ ID NOs:193-208, and the sequence of the probe that individually detects ZNF318 is selected from SEQ ID NOs:302-304. In some embodiments, the sequence of the probe that individually detects ZNF318 is SEQ ID NO:304. In some embodiments, the primer set that specifically amplifies PDK3 is a forward and reverse primer having sequences selected from SEQ ID NOs:177-192, and the sequence of the probe that individually detects PDK3 is selected from SEQ ID NOs:299-301. In some embodiments, the sequence of the probe that individually detects PDK3 is SEQ ID NO:300. In some embodiments, the primer set that specifically amplifies HMGN1 is a forward and reverse primer having sequences selected from SEQ ID NOs:209-220, and the sequence of the probe that individually detects HMGN1 is selected from SEQ ID NOs:305-307. In some embodiments, the sequence of the probe that individually detects HMGN1 is SEQ ID NO:305 In some embodiments, the primer set that specifically amplifies PTK2 is a forward and reverse primer having sequences selected from SEQ ID NOs:1-24, and the sequence of the probe that individually detects PTK2 is selected from SEQ ID NOs:253-258. In some embodiments, the sequence of the probe that individually detects PTK2 is SEQ ID NO:253. In some embodiments, the primer set that specifically amplifies SSBP2 is a forward and reverse primer having sequences selected from SEQ ID NOs:161-176, and the sequence of the probe that individually detects SSBP2 is selected from SEQ ID NOs:297 and 298. In some embodiments, the sequence of the probe that specifically detects SSBP2 is SEQ ID NO:297. In some embodiments, the primer set that specifically amplifies BCL6 is a forward and reverse primer having sequences selected from SEQ ID NOs:49-64, and the sequence of the probe that individually detects BCL6 is selected from SEQ ID NO:266-268. In some embodiments, the probe that individually detects BCL6 is SEQ ID NO:266. In some embodiments, the primer set that specifically amplifies LRMP is a forward and reverse primer having sequences selected from SEQ ID NOs:25-48, and the sequence of the probe that individually detects LRMP is selected from SEQ ID NOs:259-265. In some embodiments, the sequence of the probe that individually detects LRMP is SEQ ID NO:262. In some embodiments, the primer set that specifically amplifies ARIDA3A is a forward and reverse primer having sequences selected from SEQ ID NOs:81-96, and the sequence of the probe that individually detects ARIDA3A is selected from SEQ ID NOs:276-280. In some embodiments the sequence of the probe that individually detects ARIDA3A is SEQ ID NO:279. In some embodiments, the primer set that specifically amplifies CCND2 is a forward and reverse primer having sequences selected from SEQ ID NOs:97-112, and the sequence of the probe that individually detects CCND2 is selected from SEQ ID NOs:281-283. In some embodiments, the sequence of the probe that individually detects CCND2 is SEQ ID NO:281. In some embodiments, the primer set that specifically amplifies FOXP1 is a forward and reverse primer having sequences selected from SEQ ID NOs:221-236, and the sequence of the probe that individually detects FOXP1 is selected from SEQ ID NOs:308 and 309. In some embodiments, the sequence of the probe that individually detects FOXP1 is SEQ ID NO:309. In some embodiments, the primer set that specifically amplifies KIAA0226L is a forward and reverse primer having sequences selected from SEQ ID NOs:237-252, and the sequence of the probe that individually detects KIAA0226L is selected from SEQ ID NOs:310-314. In some embodiments, the sequence of the probe that individually detects KIAA0226L is SEQ ID NO:313. In some embodiments, the primer set that specifically amplifies JADE3 is a forward and reverse primer having sequences selected from SEQ ID NOs:145-160, and the sequence of the probe that individually detects JADE3 is selected from SEQ ID NOs:290-296. In some embodiments, the sequence of the probe that individually detects JADE3 is SEQ ID NO:292. In some embodiments, the primer set that specifically amplifies PIM2 is a forward and reverse primer having sequences selected from SEQ ID NOs:65-80, and the sequence of the probe that individually detects PIM2 is selected from SEQ ID NOs:269-275. In some embodiments, the sequence of the probe that individually detects PIM2 is SEQ ID NO:275. In some embodiments, the primer set that specifically amplifies TCF4 is a forward and reverse primer having sequences selected from SEQ ID NOs:129-144, and the sequence of the probe that individually detects TCF4 is selected from SEQ ID NOs:287-289. In some embodiments, the sequence of the probe that individually detects TCF4 is SEQ ID NO:287. In some embodiments, the primer set that specifically amplifies FAM46C is a forward and reverse primer having sequences selected from SEQ ID NOs:113-128, and the sequence of the probe that individually detects FAM46C is selected from SEQ ID NOs:284-286. In some embodiments, the sequence of the probe that individually detects FAM46C is SEQ ID NO:284.

### DETAILED DESCRIPTION OF THE INVENTION

### I. Introduction

Provided herein is a novel multiplex real-time, quantitative reverse transcription (qRT)-PCR classifier to determine cell-of-origin (COO) subtype of Diffuse Large B-cell Lymphoma (DLBCL). The classifier uses a qRT-PCR multiplex reaction to quantify 16 gene targets (15 determinative and 1 control) and assign a COO subtype of DLBCL. In some embodiments, the assay is a five-tube qRT-PCR. The feasibility and accuracy of the qRT-PCR classifier in formalin-fixed paraffin embedded tissues (FFPET) from DLBCL is shown herein.

The presently described assays rely on proven, widely adopted technology and provide accurate, reproducible, and rapid results.

### II. Definitions

The term “multiplex” refers to an assay in which more than one target is detected.

The terms “receptacle,” “vessel,” “tube,” “well,” “chamber,” “microchamber,” etc. refer to a container that can hold reagents or an assay. If the receptacle is in a kit and holds reagents, or is being used for an amplification reaction, it can be closed or sealed to avoid contamination or evaporation. If the receptacle is being used for an assay, it can be open or accessible, at least during set up of the assay.

The terms “individually detected” or “individual detection,” referring to a marker gene or marker gene product, indicates that each marker in a multiplex reaction is detected. That is, each marker is associated with a different label (detected by a differently labeled probe).

Unless otherwise labeled, the terms “COO classifier,” “subtype classifier,” “COO subtype signature,” “subtype determination signature,” and like terms are used to refer to the 15-gene signature that can be used to classify the cell of origin subtype of a DLBCL patient. The terms “6-gene COO classifier,” “6-gene subtype classifier,” “6-gene COO subtype signature,” “6-gene subtype determination signature,” and like terms refer to the classifier that includes CCND2, FOXP1, JADE3, ZNF318, SSBP2, and PTK2.

The terms “nucleic acid,” “polynucleotide,” and “oligonucleotide” refer to polymers of nucleotides (e.g., ribonucleotides or deoxyribo-nucleotides) and includes naturally-occurring (adenosine, guanidine, cytosine, uracil and thymidine), non-naturally occurring, and modified nucleic acids. The term is not limited by length (e.g., number of monomers) of the polymer. A nucleic acid may be single-stranded or double-stranded and will generally contain 5′-3′ phosphodiester bonds, although in some cases, nucleotide analogs may have other linkages. Monomers are typically referred to as nucleotides. The term “non-natural nucleotide” or “modified nucleotide” refers to a nucleotide that contains a modified nitrogenous base, sugar or phosphate group, or that incorporates a non-natural moiety in its structure. Examples of non-natural nucleotides include dideoxynucleotides, biotinylated, aminated, deaminated, alkylated, benzylated and fluorophor-labeled nucleotides.

The term “primer” refers to a short nucleic acid (an oligonucleotide) that acts as a point of initiation of polynucleotide strand synthesis by a nucleic acid polymerase under suitable conditions. Polynucleotide synthesis and amplification reactions typically include an appropriate buffer, dNTPs and/or rNTPs, and one or more optional cofactors, and are carried out at a suitable temperature. A primer typically includes at least one target-hybridized region that is at least substantially complementary to the target sequence (e.g., having 0, 1, 2, or 3 mismatches). This region of is typically about 8 to about 40 nucleotides in length, e.g., 12-25 nucleotides. A “primer set” refers to a forward and reverse primer that are oriented in opposite directions relative to the target sequence, and that produce an amplification product in amplification conditions. The primer set can further include and additional forward or reverse primer, e.g., to carry out allele-specific amplification.

As used herein, “probe” means any molecule that is capable of selectively binding to a specifically intended target biomolecule, for example, a nucleic acid sequence of interest that hybridizes to the probes. The probe is detectably labeled with at least one non-nucleotide moiety. In some embodiments, the probe is labeled with a fluorophore and quencher.

The words “complementary” or “complementarity” refer to the ability of a nucleic acid in a polynucleotide to form a base pair with another nucleic acid in a second polynucleotide. For example, the sequence A-G-T (A-G-U for RNA) is complementary to the sequence T-C-A (U-C-A for RNA). Complementarity may be partial, in which only some of the nucleic acids match according to base pairing, or complete, where all the nucleic acids match according to base pairing. A probe or primer is considered “specific for” a target sequence if it is at least partially complementary to the target sequence. Depending on the conditions, the degree of complementarity to the target sequence is typically higher for a shorter nucleic acid such as a primer (e.g., greater than 80%, 90%, 95%, or higher) than for a longer sequence.

The term “specifically amplifies” indicates that a primer set amplifies a target sequence more than non-target sequence at a statistically significant level. The term “specifically detects” indicates that a probe will detect a target sequence more than non-target sequence at a statistically significant level. As will be understood in the art, specific amplification and detection can be determined using a negative control, e.g., a sample that includes the same nucleic acids as the test sample, but not the target sequence or a sample lacking nucleic acids. For example, primers and probes that specifically amplify and detect a target sequence result in a Ct that is readily distinguishable from background (non-target sequence), e.g., a Ct that is at least 2, 3, 4, 5, 5-10, 10-20, or 10-30 cycles less than background.

The terms “identical” or “percent identity,” in the context of two or more nucleic acids, or two or more polypeptides, refer to two or more sequences or subsequences that are the same or have a specified percentage of nucleotides, or amino acids, that are the same (e.g., about 60% identity, e.g., at least any of 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” Percent identity is typically determined over optimally aligned sequences, so that the definition applies to sequences that have deletions and/or additions, as well as those that have substitutions. The algorithms commonly used in the art account for gaps and the like. Typically, identity exists over a region comprising an a sequence that is at least about 8-25 amino acids or nucleotides in length, or over a region that is 50-100 amino acids or nucleotides in length, or over the entire length of the reference sequence.

The term “kit” refers to any manufacture (e.g., a package or a container) including at least one reagent, such as a nucleic acid probe or probe pool or the like, for specifically amplifying, capturing, tagging/converting or detecting RNA or DNA as described herein.

The term “amplification conditions” refers to conditions in a nucleic acid amplification reaction (e.g., PCR amplification) that allow for hybridization and template-dependent extension of the primers. The term “amplicon” or “amplification product” refers to a nucleic acid molecule that contains all or a fragment of the target nucleic acid sequence and that is formed as the product of in vitro amplification by any suitable amplification method. One of skill will understand that a forward and reverse primer (primer pair) defines the borders of an amplification product. The term “generate an amplification product” when applied to primers, indicates that the primers, under appropriate conditions (e.g., in the presence of a nucleotide polymerase and NTPs), will produce the defined amplification product. Various PCR conditions are described in PCR Strategies (Innis et al., 1995, Academic Press, San Diego, CA) at Chapter 14; PCR Protocols: A Guide to Methods and Applications (Innis et al., Academic Press, N Y, 1990)

The term “amplification product” refers to the product of an amplification reaction. The amplification product includes the primers used to initiate each round of polynucleotide synthesis. An “amplicon” is the sequence targeted for amplification, and the term can also be used to refer to amplification product. The 5′ and 3′ borders of the amplicon are defined by the forward and reverse primers.

The term “sample” or “biological sample” refers to any composition containing or presumed to contain nucleic acid. The term includes purified or separated components of cells, tissues, or blood, e.g., DNA, RNA, proteins, cell-free portions, or cell lysates. In the context of the presently disclosed assay, the sample is typically FFPET, e.g., from a tumor or metastatic lesion. The sample can also be from frozen or fresh tissue, or from a liquid sample, e.g., blood or a blood component (plasma or serum), urine, semen, saliva, sputum, mucus, semen, tear, lymph, cerebral spinal fluid, mouth/throat rinse, bronchial alveolar lavage, material washed from a swab, etc. Samples also may include constituents and components of in vitro cultures of cells obtained from an individual, including cell lines. The sample can also be partially processed from a sample directly obtained from an individual, e.g., cell lysate or blood depleted of red blood cells.

The term “obtaining a sample from an individual” means that a biological sample from the individual is provided for testing. The obtaining can be directly from the individual, or from a third party that directly obtained the sample from the individual.

A “control” sample or value refers to a value that serves as a reference, usually a known reference, for comparison to a test sample or test conditions. For example, a test sample can be taken from a test condition, e.g., from an individual suspected of having cancer, and compared to samples from known conditions, e.g., from a cancer-free individual (negative control), or from an individual known to have cancer (positive control). In the context of the present disclosure, the test sample is typically from a DLBCL patient. A control can also represent an average value or a range gathered from a number of tests or results. A control can also be prepared for reaction conditions. For example, a control for the presence, quality, and/or quantity of nucleic acid (e.g., internal control) can include primers or probes that will detect a sequence known to be present in the sample (e.g., a housekeeping gene such as beta actin, beta globin, glyceraldehyde 3-phosphate dehydrogenase (GAPDH), ribosomal protein L37 and L38, PPIase, EIF3, eukaryotic translation elongation factor 2 (eEF2), DHFR, or succinate dehydrogenase) A known added polynucleotide, e.g., having a designated length, can also be added. An example of a negative control is one free of nucleic acids, or one including primers or probes specific for a sequence that would not be present in the sample, e.g., from a different species. One of skill will understand that the selection of controls will depend on the particular assay, e.g., so that the control is cell type and organism-appropriate. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “label,” “tag,” “detectable moiety,” and like terms refer to a composition detectable by spectroscopic, photochemical, biochemical, immunochemical, chemical, or other physical means. For example, useful labels include fluorescent dyes (fluorophores), luminescent agents, radioisotopes (e.g., ³²P, ³H), electron-dense reagents, or an affinity-based moiety, e.g., a poly-A (interacts with poly-T) or poly-T tag (interacts with poly-A), a His tag (interacts with Ni), or a strepavidin tag (separable with biotin).

The term “identifying an individual” means determining based on a sample derived from an individual (e.g., a patient) whether the respective individual is actually sensitive to an administration or treatment.

The term “providing treatment for an individual” means that the treatment is actually administered to the individual (e.g., an in-patient injection), or that it is made available to the individual, so that the individual or third party actually administers the treatment.

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4th ed. 2007); Sambrook et al., MOLECULAR CLONING, A LABORATORY MANUAL, Cold Springs Harbor Press (Cold Springs Harbor, N.Y. 1989). The term “a” or “an” is intended to mean “one or more.” The terms “comprise,” “comprises,” and “comprising,” when preceding the recitation of a step or an element, are intended to mean that the addition of further steps or elements is optional and not excluded.

### III. Nucleic Acid Samples

Samples for nucleic acid amplification can be obtained from any source suspected of containing nucleic acid. Samples can be taken from formalin fixed paraffin embedded tissue (FFPET), tissue biopsy, brochoalveolar lavage, or cultured cells (e.g., obtained from a patient, or representing a control). In the context of the present disclosure, the sample is typically taken from lung tissue or a cell population that includes lung cells, e.g., lung cancer cells. In some embodiments, the sample is obtained in a non-invasive manner, e.g., from urine, skin, swab, saliva, blood or a blood fraction.

In a sample that includes cells, the cells can be separated out (e.g., using size-based filtration or centrifugation), thereby leaving cell free nucleic acids (cfNA), including nucleic acids in exosomes, microvesicles, viral particles, or those circulating freely. Alternatively, the cells can be lysed to obtain cellular nucleic acids, either in the presence of magnetic glass particles (MGPs) or before addition of the cellular lysate to the MGPs.

Methods for isolating nucleic acids from biological samples are known, e.g., as described in Sambrook, and several kits are commercially available (e.g., High Pure RNA Isolation Kit, High Pure Viral Nucleic Acid Kit, and MagNA Pure LC Total Nucleic Acid Isolation Kit, DNA Isolation Kit for Cells and Tissues, DNA Isolation Kit for Mammalian Blood, High Pure FFPET DNA Isolation Kit, available from Roche). In the context of the presently disclosed methods, RNA is collected, though in some embodiments, the classifier can be used on previously prepared cDNA.

### IV. Diffuse Large B Cell Lymphoma (DLBCL) and Therapies

Diffuse large B-cell lymphoma (DLBCL) is the most common subtype of non-Hodgkin lymphoma. Approximately 40% of patients have refractory disease or disease that will relapse after an initial response, and the majority of patients with relapsed DLBCL will succumb to the disease. There are two major biologically distinct molecular subtypes of DLBCL: germinal center B-cell (GCB) and activated B-cell (ABC). ABC DLBCL is associated with substantially worse outcomes when treated with standard chemotherapy.

GCB patients typically benefit from standard chemotherapy. This can include CHOP (cyclophosphamide; doxorubicin; vincristine; and prednisolone) or R-CHOP, which further includes rituximab and/or etoposide. The cocktail can be administered periodically for a set period of time, or until reduction in tumor size and/or symptoms are detected. For example, the CHOP or R-CHOP can be administered every 2 or 3 weeks. Treatment or administration typically begins with a low dose so that side effects can be determined, and the dose increased until side effects appear or within the patient's tolerance.

A number of additional agents (alternative therapies) are in development for ABC patients. These can be administered in combination with CHOP or R-CHOP, simultaneously or in separate doses. These alternative therapies can include BTK inhibitors (e.g., ibrutinib), SYK inhibitors (e.g., fostamatinib), NFkB inhibitors (e.g., bortezomib), or immunomodulatory agents (e.g., structural and functional analogs of thalidomide, e.g., lenalidomide).

Additional appropriate therapies for DLBCL GCB and ABC subtypes are described in Dunleavy et al. (Apr. 15, 2014) Oncology and Nowakowki & Czuczman (2015) Am. Soc. Clin. Oncol. Educ. Book e449.

### V. Amplification and Detection

A nucleic acid sample can be used for detection and quantification, e.g., using nucleic acid amplification, e.g., using any primer-dependent method. In some embodiments, a preliminary reverse transcription step is carried out (also referred to as RT-PCR, not to be confused with real time PCR). See, e.g., Hierro et al. (2006) 72:7148. The term “qRT-PCR” as used herein refers to reverse transcription followed by quantitative PCR. Both reactions can be carried out in a single tube without interruption, e.g., to add reagents. For example, a polyT primer can be used to reverse transcribe all mRNAs in a sample with a polyA tail, random oligonucleotides can be used, or a primer can be designed that is specific for a particular target transcript that will be reverse transcribed into cDNA. The cDNA can form the initial template strand to be for quantitative amplification (real time or quantitative PCR, i.e., RTPCR or qPCR). qPCR allows for reliable detection and measurement of products generated during each cycle of PCR process. Such techniques are well known in the art, and kits and reagents are commercially available, e.g., from Roche Molecular Systems, Life Technologies, Bio-Rad, etc. See, e.g., Pfaffl (2010) Methods: The ongoing evolution of qPCR vol. 50.

A separate reverse transcriptase and thermostable DNA polymerase can be used, e.g., in a two-step (reverse transcription followed by addition of DNA polymerase and amplification) or combined reaction (with both enzymes added at once). In some embodiments, the target nucleic acid is amplified with a thermostable polymerase with both reverse transcriptase activity and DNA template-dependent activity. Exemplary enzymes include Tth DNA polymerase, the C. therm Polymerase system, and those disclosed in US20140170730 and US20140051126.

Probes for use as described herein can be labeled with a fluorophore and quencher (e.g., TaqMan, LightCycler, Molecular Beacon, Scorpion, or Dual Labeled probes). Appropriate fluorophores include FAM, JOE, TET, Cal Fluor Gold 540, HEX, VIC, Cal Fluor Orang 560, TAMRA, Cyanine 3, Quasar 570, Cal Fluor Red 590, Rox, Texas Red, Cyanine 5, Quasar 670, and Cyanine 5.5. Appropriate quenchers include TAMRA (for FAM, JOE, and TET), DABCYL, and BHQ1-3.

Detection devices are known in the art and can be selected as appropriate for the selected labels. Detection devices appropriate for quantitative PCR include the Cobas® and Light Cycler© systems (Roche), PRISM 7000 and 7300 real-time PCR systems (Applied Biosystems), etc. Six-channel detection is available on the CFX96 Real Time PCR Detection System (Bio-Rad) and Rotorgene Q (Qiagen), allowing for a higher degree of multiplexing.

Results can be expressed in terms of a threshold cycle (abbreviated as Ct, and in some instances Cq or Cp). A lower Ct value reflects the rapid achievement of a predetermined threshold level, e.g., because of higher target nucleic acid concentration or a more efficient amplification. A higher Ct value may reflect lower target nucleic acid concentration, or inefficient or inhibited amplification. The threshold cycle is generally selected to be in the linear range of amplification for a given target. In some embodiments, the Ct is set as the cycle at which the growth signal exceeds a pre-defined threshold line, e.g., in relation to the baseline, or by determining the maximum of the second derivation of the growth curve. Determination of Ct is known in the art, and described, e.g., in U.S. Pat. No. 7,363,168.

### VI. Kits

Provided herein are kits for multiplex qRT-PCR assays to classify the COO subtype of a DLBCL patient. In some embodiments, the kit includes mixtures of primers and probes for amplification, detection, and quantification of GCB and ABC marker gene products (RNA). GCB markers include ZNF318, PDK3, HMGN1, PTK2, SSBP2, BCL6, and LRMP, and transcripts of these genes are present at a higher level in samples from GCB patients than in samples from non-cancer or ABC patients. ABC markers include ARID3A, CCND2, FOXP1, KIAA0226L, JADE3, PIM2, TCF4, and FAM46C, and transcripts of these genes are present at a higher level in samples from ABC patients than in samples from non-cancer or GCB patients.

Kits for multiplex qRT-PCR assays to classify the 6-gene COO subtype of a DLBCL patient are also included herein. In some embodiments, the kit includes mixtures of primers and probes for amplification, detection, and quantification of GCB and ABC marker gene products (RNA). GCB markers include ZNF318, PTK2, and SSBP2, and transcripts of these genes are present at a higher level in samples from GCB patients than in samples from non-cancer or ABC patients. ABC markers include CCND2, FOXP1, and JADE3, transcripts of these genes are present at a higher level in samples from ABC patients than in samples from non-cancer or GCB patients.

The marker-specific primer sets and probes can be mixed and matched in any combination. For example, each marker can be individually detected. In a detection system having 6 channels, up to 5 markers can be detected in a single vessel, along with the internal control. In this case, only 3 primer set and probe mixtures are required to include all 15 markers. In a detection system having 4 channels, up to 3 markers can be detected in a single vessel, along with the internal control. In this case, 5 primer set and probe mixtures are required. Alternatively, the assay can be carried out with a lower degree of multiplexing, or in non-multiplex fashion, so that more primer set and probe mixtures are required to test expression of all 15 markers in a sample. An example of a 5-tube multiplex assay is shown in the Examples. Thus in some embodiments, the kit includes 5 mixtures (e.g., master mixes), each comprising a primer set and probe specific for up to three GCB and ABC probe markers, and a primer set and probe specific for an internal control gene.

For the 6-gene COO signature, the kit can include 2 mixtures, e.g., (i) a mixture including primers and probes that specifically amplify and detect GCB markers include ZNF318, PTK2, and SSBP2 (and an internal control) and (ii) a mixture including primers and probes that specifically amplify and detect ABC markers include CCND2, FOXP1, and JADE3 (and an internal control). In some embodiments, the probe for each gene in mixture (i) and (ii) has a different label so that the expression of each gene can be individually detected. In some embodiments, the probe for each of the determinative gene (not an internal control) in mixture (i) and (ii) has the same label. In some embodiments, the kit includes 6 different mixtures, one for each gene in the 6-gene COO signature.

In some embodiments, markers are not detected individually. For example, all of the probes specific for GCB markers can be labeled with the same label, and all of the probes specific for ABC markers can be labeled with the same label (different from that on the GCB probes). In this case, all 15 markers can be massively multiplexed in a single vessel for detection with only 3 channels (one for GCB marker probes, one for ABC marker probes, and one for the control probe).

In some embodiments, the mixtures further comprise buffers, dNTPs, and other elements (e.g., cofactors or aptamers) appropriate for reverse transcription and amplification. Typically, the mixture is concentrated, so that an aliquot is added to the final reaction volume, along with sample (e.g., RNA), enzymes, and/or water. In some embodiments, the kit further comprises reverse transcriptase (or an enzyme with reverse transcriptase activity), and/or DNA polymerase (e.g., thermostable DNA polymerase such as Taq, ZOS, and derivatives thereof).

In some embodiments, the kit further includes components for RNA purification from a sample, e.g., an FFPET sample. For example, the kit can include components from High Pure or MagNA Pure FFPE RNA Isolation Kits (Roche), RNeasy FFPE Kit (Qiagen), PureLink FFPE RNA Isolation Kit (Thermo Fisher), etc.

In some embodiments, the kit further includes at least one control sample, e.g., nucleic acids from non-cancer sample (or pooled samples), or from a known ABC or GCB sample (or pooled samples). In some embodiments, the kit includes an ABC positive control and/or a GCB positive control. In some embodiments, the kit includes a negative control, e.g., lacking nucleic acids, or lacking ABC and/or GCB marker nucleic acids. In some embodiments, the kit further includes consumables, e.g., plates or tubes for nucleic acid preparation, tubes for sample collection, etc. In some embodiments, the kit further includes instructions for use, reference to a website, or software.

### VII. Examples

### Design of COO Subtype Determination Signature

A set of commercially acquired DLBCL FFPET specimens (Training cohort 1; n=32) was used to select the classifier genes (Table 1). The samples were prepared using the FFPET RNA kit from Roche.

Gene targets in the qRT-PCR classifier were derived from a collection of genes (n=76) screened in a cohort of DLBCL specimens (n=32; Training cohort). We used the Affymetrix microarray platform as a “gold standard” for confirmation.

TABLE 1
qRT-PCR COO classifier for DLBCL with GenBank Accession Numbers

| GCB genes | ABC genes | Control (reference) gene |
|---|---|---|
| ZNF318 | ARIDA3A | Internal control (IC) |
| Zinc finger protein 318 | AT-rich interaction domain 3A | |
| CH471081.1 (GeneID 24149) | CH471139.2 (GeneID 1820) | |
| PDK3 | CCND2 | |
| Pyruvate dehydrogenase kinase 3 | Cyclin D2 | |
| C471074 (GeneID 5165) | CH471116.2 (GeneID 894) | |
| HMGN1 | FOXP1 | |
| High mobility group nucleosome | Forkhead box P1 | |
| binding domain 1 | CH471055.1 (GeneID 27086) | |
| CH471079.2 (GeneID 3150) | | |
| PTK2 | KIAA0226L | |
| Protein tyrosine kinase 2 | KIAA0226 like | |
| CH471060.1 (GeneID 5747) | CH471075.1 (GeneID 80183) | |
| SSBP2 | JADE3 | |
| Single stranded DNA binding | Jade family PHD finger 3 | |
| protein 2 | CH471164.1 (GeneID 9767) | |
| CH471084.1 (GeneID 23635) | | |
| BCL6 | PIM2 | |
| B cell CLL/lymphoma 6 | Pim2 serine/threonine kinase | |
| CH471052.2 (GeneID 604) | CH471224.1 (GeneID 11040) | |
| LRMP | TCF4 | |
| Lymphoid restricted membrane | Transcription factor 4 | |
| protein | CH471096.1 (GeneID 6925) | |
| CH471094.1 (GeneID 4033) | | |
| | FAM46C | |
| | Family with sequence similarity | |
| | 46 member C | |
| | CH471122.1 (GeneID 54855) | |

Once the genes were selected, a qRT-PCR assay was designed to be performed in 5 separate wells. 200 ng RNA test and control sample (40 ng/well) was used.

Reaction conditions were as follows for each reaction:

25 ul RNA+25 ul reaction mix

Reaction mix: 5 ul manganese acetate+10 ul RNA master mix stock+10 ul primer/probe mix (final concentration 100-300 nM)

Reactions were run in a Cobas® LC480 with four filters to detect probes as indicated in Table 2.

TABLE 2
Exemplary assay lay-out

| Label | Well 1 | Well 2 | Well 3 | Well 4 | Well 5 |
|---|---|---|---|---|---|
| FAM | ARID3A | TCF4 | PDK3 | SSBP2 | JADE3 |
| HEX | CCND2 | ZNF318 | HMGN1 | BCL6 | PIM2 |
| JA270 | FOXP1 | KIAA0226L | PTK2 | LRMP | FAM46C |
| CY5.5 | IC | IC | IC | IC | IC |

Table 2 shows an exemplary assay design, and allows individual detection and quantification of each of the marker genes in a minimal number of wells.

More or fewer reaction vessels can be used. For example, a one-tube assay having all of the GCB markers labeled with the same label (fluorophore 1), all of the ABC markers labeled with the same label (fluorophore 2), and an internal control (IC) labeled with a different label (fluorophore 3) can be used. On the other end of the spectrum, each classifier gene can be detected in a separate well to determine the COO subtype of the test sample. The test is carried out by comparing the expression level of the GCB markers to the expression level of the ABC markers within a sample. If the ratio of GCB marker expression to ABC marker expression is higher than a threshold (e.g., GCB threshold), the result indicates that the sample is from an individual with GCB DLBCL. If the ratio of ABC marker expression to GCB marker expression is higher than a threshold (e.g., ABC threshold), the result indicates that the sample is from an individual with ABC DLBCL. The internal control is used to standardize expression levels based on the amount or quality of nucleic acid in the sample.

The threshold levels are based on the probability that the GCB and ABC expression levels in a sample from an individual accurately classify the individual's DLBCL COO subtype. For example, a GCB threshold level can be set using a sample from an individual (or group of individuals) known to have GCB subtype. A GCB positive control can then be prepared with the known GCB sample. In some embodiments, the GCB positive control is prepared from the known GCB sample mixed with a sample known to be from an individual with ABC so that >50% of the nucleic acids in the GCB positive control are from the known GCB sample to provide a minimum GCB:ABC expression level ratio. If a sample has a GCB:ABC expression ratio above that ratio (GCB threshold), the result is considered an accurate call of GCB COO subtype. The GCB positive control can be prepared with 51-100% known GCB sample, e.g., about 55, 58, 60, 62, 65, 68, 70, 75% or higher, with higher percentages resulting in a more stringent confidence level in the GCB threshold. If a sample has a GCB:ABC expression ratio below the GCB threshold, the result is either not determined, or an ABC COO subtype. The ABC threshold is set similarly. For example, the ABC positive control can be prepared with 51-100% known ABC sample, e.g., about 55, 58, 60, 62, 65, 68, 70, 75% or higher, with higher percentages resulting in a more stringent confidence level in the ABC threshold. If a sample has an ABC:GCB expression ratio above the ABC threshold, the result is considered an accurate call of ABC COO subtype, while a ratio below the ABC threshold, the result is not determined, or a GCB COO subtype. In some embodiments, the GCB and ABC positive controls are prepared by mixing a known amount of GCB marker nucleic acids and ABC marker nucleic acids. The GCB and ABC positive controls also act as controls for assay performance, e.g., to ensure reagents are added and that the instrument is performing properly.

Tables 3 and 4 show the sequences of primers and probes, respectively, that can be used for the present classifier.

TABLE 3
Primer sequences

| | Forward | 5′ to 3′ Sequence | Reverse | 5′ to 3′ Sequence |
|---|---|---|---|---|
| Gene | primer | (SEQ ID NO) | Primer | (SEQ ID NO) |
| PTK2 | CS_PTK2_F1 | GGCAGTATTGACAGGGAG | CS_PTK2_R1 | TGGTTTACCCACAGGCTGA |
| | | GA (1) | | (2) |
| PTK2 | CS_PTK2_F2 | GGAGAAGGCCAATTTGGA | CS_PTK2_R2 | ACAGTTTTTACATGTTTTAAT |
| | | GAT (3) | | TGCAACC (4) |
| PTK2 | CS_PTK2_F3 | GGACAGAAAAGGAATGCT | CS_PTK2_R3 | CGCAATGGTTAGGGATGGTG |
| | | ACAACTA (5) | | (6) |
| PTK2 | CS_PTK2_F4 | AGCTTAGTACAGCTCTTG | CS_PTK2_R4 | TCATTTGAGGACACCAGAAC |
| | | CAT (7) | | ATT (8) |
| PTK2 | CS_PTK2_F5 | GCCCAGAAGAAGGAATCA | CS_PTK2_R5 | GGTTTGCACTTGAGTGAAGT |
| | | GTT (9) | | C (10) |
| PTK2 | CS_PTK2_F6 | ACCATTCCCCTCCTACCA | CS_PTK2_R6 | CTCACCCAGGTCAGAGTTCA |
| | | G (11) | | (12) |
| PTK2 | CS_PTK2_F7 | GACCTCAGGAGATAGCAA | CS_PTK2_R7 | CACTTGCCCAATCCCTCG |
| | | TGT (13) | | (14) |
| PTK2 | CS_PTK2_F8 | GAATGGAACCTCGCAGTC | CS_PTK2_R8 | GGCCAACTTTGGTATTGATG |
| | | AT (15) | | G (16) |
| PTK2 | CS_PTK2_F9 | TTCGACGTTTTACCTCAG | CS_PTK2_R9 | GGCTTCACACCATGCATCAG |
| | | CT (17) | | (18) |
| PTK2 | CS_PTK2_F10 | GAAAGAAGGTGAACGGGC | CS_PTK2_R10 | GTGTGTCCGCATGCCTTG |
| | | TT (19) | | (20) |
| PTK2 | CS_PTK2_F11 | CCTGTCTGGATAATCATG | CS_PTK2_R11 | GCTAGATCCAAACTGTATTTC |
| | | GAGC (21) | | CTTAC (22) |
| PTK2 | CS_PTK2_F12 | CGGCCCAGGTTTACTGAA | CS_PTK2_R12 | TCTTCTTGCTGAGCCTTCTCT |
| | | C (23) | | (24) |
| LRMP | LRMP_F1 | CAGGCTGCATCAGGATGA | LRMP_R1 | CAGCAGGCTCTCAGGACA |
| | | AT (25) | | (26) |
| LRMP | LRMP_F2 | CTCTCAGGCTGCATCAGG | LRMP_R1 | CAGCAGGCTCTCAGGACA |
| | | A (27) | | (28) |
| LRMP | LRMP_F3 | ACAAGATCAGCTTCTCCC | LRMP_R2 | CTCGTAGAGTCTTGGAATGC |
| | | AC (29) | | A (30) |
| LRMP | LRMP_F4 | CAAGATCAGCTTCTCCCA | LRMP_R3 | CTCGTAGAGTCTTGGAATGC |
| | | CG (31) | | AAT (32) |
| LRMP | LRMP_F5 | AGGAACCAGAAACAATAG | LRMP_R4 | CGATTTCACAGTGGTTACAG |
| | | AAGAACA (33) | | GA (34) |
| LRMP | LRMP_F6 | GCCAAAGAGGAACCAGAA | LRMP_R5 | ACCGATTTCACAGTGGTTAC |
| | | ACA (35) | | A (36) |
| LRMP | LRMP_F7 | CAGTAGGGCTGAGATGTT | LRMP_R6 | ACGTGCTGAATCATCACTTCA |
| | | GG (37) | | A (38) |
| LRMP | LRMP_F8 | GGCCAGTAGGGCTGAGAT | LRMP_R6 | ACGTGCTGAATCATCACTTCA |
| | | (39) | | A (40) |
| LRMP | LRMP_F9 | GACTGCCAAATTAAAAAA | LRMP_R7 | TTTCTGGGTAAAGAGGCAAT |
| | | CGTTCA (41) | | AGTC (42) |
| LRMP | LRMP_F10 | GGAAGCCAAGTCTTTCTG | LRMP_R8 | GAGGACTTGAGATTTGTTGC |
| | | AAAAG (43) | | C (44) |
| LRMP | LRMP_F11 | GGCAGAAGAAAATTTGAA | LRMP_R9 | AATGATTTCCTGTGCCTGGT |
| | | GAAAGAAA (45) | | (46) |
| LRMP | LRMP_F12 | GGTGGCTGGGATGGAAAATAA | LRMP_R10 | GGACGGTGTTCACTCTGCT |
| | | (47) | | (48) |
| BCL6 | BCL6_F1 | GAAGAGCCACCTGCGAATC | BCL6_R1 | GCTGGCTTTTGTGACGGAAA |
| | | (49) | | (50) |
| BCL6 | BCL6_F2 | CCACCTGCGAATCCACAC (51) | BCL6_R2 | CTGGCTTTTGTGACGGAAAT |
| | | | | G (52) |
| BCL6 | BCL6_F1 | GAAGAGCCACCTGCGAATC | BCL6_R3 | GCTGGCTTTTGTGACGGAA |
| | | (53) | | (54) |
| BCL6 | BCL6_F2 | CCACCTGCGAATCCACAC (55) | BCL6_R1 | GCTGGCTTTTGTGACGGAAA |
| | | | | (56) |
| BCL6 | BCL6_F3 | CATGTTGTGGACACTTGCC | BCL6_R4 | CTTCACGAGGAGGCTTGATG |
| | | (57) | | (58) |
| BCL6 | BCL6_F4 | GGAGCATGTTGTGGACAC | BCL6_R5 | CTCTTCACGAGGAGGCTTGA |
| | | TT (59) | | (60) |
| BCL6 | BCL6_F5 | ATGGAGCATGTTGTGGAC | BCL6_R6 | CGAGGAGGCTTGATGGCA |
| | | AC (61) | | (62) |
| BCL6 | BCL6_F6 | GGACTCCACCATCCCACAA | BCL6_R7 | TAGAGTGGTGAGTGGCTCTC |
| | | (63) | | (64) |
| PIM2 | PIM2_FP1 | GTGCCCTGCTTCATGATG (65) | PIM2_RP1 | CTGGTGTCGAGAGATCCACT |
| | | | | C (66) |
| PIM2 | PIM2_FP2 | GGCTGTGCCAAACTCATT (67) | PIM2_RP2 | GGGCTGTACACCCTTGT (68) |
| PIM2 | PIM2_FP1 | GTGCCCTGCTTCATGATG (69) | PIM2_RP3 | CATGGTACTGGTGTCGAGAG |
| | | | | A (70) |
| PIM2 | PIM2_FP1 | GTGCCCTGCTTCATGATG (71) | PIM2_RP4 | CCGGGAGTGCATGGTACT |
| | | | | (72) |
| PIM2 | PIM2_FP3 | GGACACCGCCTCACAGAT (73) | PIM2_RP5 | AGTGGGCATGTGACTGAGTC |
| | | | | (74) |
| PIM2 | PIM2_FP3 | GGACACCGCCTCACAGAT (75) | PIM2_RP6 | CTTCGAGTGGGCATGTGA |
| | | | | (76) |
| PIM2 | PIM2_FP4 | CGCCTCACAGATCGACTC (77) | PIM2_RP7 | GCAGTGCGACTTCGAGTG |
| | | | | (78) |
| PIM2 | PIM2_FP5 | ACCGTCTTCGCAGGACAC (79) | PIM2_RP8 | GGGCATGTGACTGAGTCTG |
| | | | | (80) |
| ARID3A | ARID3A_FP1 | GGCGACTGGACTTACGAGG | ARID3A_RP1 | CCAGGAATTCCTTCCTCTTGG |
| | | (81) | | (82) |
| ARID3A | ARID3A_FP2 | TCCTGGATGACTTGTTCA | ARID3A_RP2 | AGGACCTGTTTGGCCATGAT |
| | | GC (83) | | (84) |
| ARID3A | ARID3A_FP3 | ACGTCCATCACCAGTGCA (85) | ARID3A_RP3 | CTTCTCACACTCGTAGGGGT |
| | | | | (86) |
| ARID3A | ARID3A_FP3 | ACGTCCATCACCAGTGCA (87) | ARID3A_RP4 | CCCGCTTCTCACACTCGTA |
| | | | | (88) |
| ARID3A | ARID3A_FP4 | CAGCTGCCCATGAGCATTC | ARID3A_RP5 | TCAGGTTCACAGCAGAGTCC |
| | | (89) | | (90) |
| ARID3A | ARID3A_FP4 | CAGCTGCCCATGAGCATTC | ARID3A_RP6 | CGTCAGGTTCACAGCAGAG |
| | | (91) | | (92) |
| ARID3A | ARID3A_FP5 | AGCATGTCGGTGGAGATC | ARID3A_RP7 | TTGGGAGCAGAGGTTGGC |
| | | AA (93) | | (94) |
| ARID3A | ARID3A_FP6 | ATCAGCATGTCGGTGGAG | ARID3A_RP8 | TTTGTTGGGAGCAGAGGTTG |
| | | AT (95) | | (96) |
| CCND2 | CCND2_FP1 | AGGACATCCAACCCTACATGC | CCND2_RP1 | GAAGACCTCTTCTTCGCACT |
| | | (97) | | (98) |
| CCND2 | CCND2_FP2 | CTTCATTGCTCTGTGTGCCA | CCND2_RP2 | TGCTCCCACACTTCCAGTT |
| | | (99) | | (100) |
| CCND2 | CCND2_FP2 | CTTCATTGCTCTGTGTGCCA | CCND2_RP3 | CCACACTTCCAGTTGCGATC |
| | | (101) | | (102) |
| CCND2 | CCND2_FP3 | GACTGAGCTGCTGGCTAAGA | CCND2_RP4 | GAGCACCGCCTCAATCTG |
| | | (103) | | (104) |
| CCND2 | CCND2_FP4 | GGACATCCAACCCTACATGC | CCND2_RP5 | AGAGGGAAGACCTCTTCTTC |
| | | (105) | | G (106) |
| CCND2 | CCND2_FP4 | GGACATCCAACCCTACATGC | CCND2_RP6 | GGAAGACCTCTTCTTCGCACT |
| | | (107) | | (108) |
| CCND2 | CCND2_FP2 | CTTCATTGCTCTGTGTGCCA | CCND2_RP7 | CTGCTCCCACACTTCCAGT |
| | | (109) | | (110) |
| CCND2 | CCND2_FP5 | ACCTTCATTGCTCTGTGTGC | CCND2_RP8 | GCTCCCACACTTCCAGTTG |
| | | (111) | | (112) |
| FAM46C | FAM46C_FP1 | AAGGACCTGCCTCTGTCG | FAM46C_RP1 | TCTCCTCTGCCATCTTCAGG |
| | | (113) | | (114) |
| FAM46C | FAM46C_FP2 | CCAAGGACCTGCCTCTGT | FAM46C_RP2 | CTCCTCTGCCATCTTCAGGG |
| | | (115) | | (116) |
| FAM46C | FAM46C_FP3 | CCAAGGACCTGCCTCTGTC | FAM46C_RP1 | TCTCCTCTGCCATCTTCAGG |
| | | (117) | | (118) |
| FAM46C | FAM46C_FP4 | CCAAGGACCTGCCTCTGTC | FAM46C_RP2 | CTCCTCTGCCATCTTCAGGG |
| | | (119) | | (120) |
| FAM46C | FAM46C_FP5 | CAAGGACCTGCCTCTGTCG | FAM46C_RP1 | TCTCCTCTGCCATCTTCAGG |
| | | (121) | | (122) |
| FAM46C | FAM46C_FP2 | CCAAGGACCTGCCTCTGT | FAM46C_RP3 | CTCTGCCATCTTCAGGGGAT |
| | | (123) | | (124) |
| FAM46C | FAM46C_FP1 | AAGGACCTGCCTCTGTCG | FAM46C_RP4 | TGCTCTCCTCTGCCATCTTC |
| | | (125) | | (126) |
| FAM46C | FAM46C_FP3 | CCAAGGACCTGCCTCTGTC | FAM46C_RP5 | CTCTGCCATCTTCAGGGGAT |
| | | (127) | | (128) |
| TCF4 | TCF4_FP1 | AAACCAGCAACCAGCACTTT | TCF4_RP1 | GAGGAGCTCCAAGGGTCAC |
| | | (129) | | (130) |
| TCF4 | TCF4_FP2 | CAACCAGCACTTTCCCTAGC | TCF4_RP2 | CCACTGGAGGAGCTCCAAG |
| | | (131) | | (132) |
| TCF4 | TCF4_FP3 | ACTTCCCCTGACCTGAACC | TCF4_RP3 | GAGACACTCTGCCCCTGTAG |
| | | (133) | | (134) |
| TCF4 | TCF4_FP4 | CGACTTCCCCTGACCTGAA | TCF4_RP4 | GAGACACTCTGCCCCTGTAG |
| | | (135) | | (136) |
| TCF4 | TCF4_FP5 | GCAACCAGCACTTTCCCTAG | TCF4_RP5 | ATTCATCCCACTGGAGGAGC |
| | | (137) | | (138) |
| TCF4 | TCF4_FP6 | TGGCCGTCATCCTCAGTC | TCF4_RP6 | CCTCCCTTCTTTTCAGACACG |
| | | (139) | | (140) |
| TCF4 | TCF4_FP7 | GTGGCCGTCATCCTCAGTC | TCF4_RP7 | TCTTTTCAGACACGCAGCTT |
| | | (141) | | (142) |
| TCF4 | TCF4_FP8 | GTGGCCGTCATCCTCAGT | TCF4_RP7 | TCTTTTCAGACACGCAGCTT |
| | | (143) | | (144) |
| JADE3 | JADE3_FP1 | TCAAGTCAAAAATTCCAAATG | JADE3_RP1 | TGGAAGTTTCATGGCACTGA |
| | | AACAC (145) | | (146) |
| JADE3 | JADE3_FP2 | TACCAGCCAGTCCAGACAC | JADE3_RP2 | TCGGATAAACAGAACGTCCT |
| | | (147) | | T (148) |
| JADE3 | JADE3_FP3 | GGCTTCAGGAACTCAATGAAGA | JADE3_RP3 | CAGGACTTCTACTGTCTTTTC |
| | | (149) | | CA (150) |
| JADE3 | JADE3_FP3 | GGCTTCAGGAACTCAATGAAGA | JADE3_RP4 | GGCGTTCCAGGACTTCTACT |
| | | (151) | | (152) |
| JADE3 | JADE3_FP4 | GGGAATGATATGGTGTTCTGTGA | JADE3_RP5 | CCTTCTGGGACCTTGAGGA |
| | | (153) | | (154) |
| JADE3 | JADE3_FP5 | ACTAAATGGGCTCATGTCAGC | JADE3_RP6 | ATCGGTTCCATCCTCTCAGG |
| | | (155) | | (156) |
| JADE3 | JADE3_FP6 | TGAGAATGTTTATGCATCTACGC | JADE3_RP7 | AGCTTCTCTCGTCTGCTTATC |
| | | (157) | | A (158) |
| JADE3 | JADE3_FP7 | GGTTTGCAAGTCCAGCTTCT | JADE3_RP8 | TGGTGGGTAAAACAGTGAGT |
| | | (159) | | TT (160) |
| SSBP2 | SSBP2__F1 | GCCACCAGGAACACCCAT | SSBP2_R1 | GGTCCAGGAGGTACTGCATT |
| | | (161) | | (162) |
| SSBP2 | SSBP2_F2 | ACATGTATACTTTAATGAATGC | SSBP2_R2 | TGGGACCATCTGACCCAG |
| | | AGTACC (163) | | (164) |
| SSBP2 | SSBP2_F3 | AACATGTATACTTTAATGAATG | SSBP2_R3 | ATGGGACCATCTGACCCAG |
| | | CAGTACC (165) | | (166) |
| SSBP2 | SSBP2_F1 | GCCACCAGGAACACCCAT | SSBP2_R4 | CCAGGAGGTACTGCATTCAT |
| | | (167) | | T (168) |
| SSBP2 | SSBP2_F1 | GCCACCAGGAACACCCAT | SSBP2_R5 | GTCCAGGAGGTACTGCATTC |
| | | (169) | | A (170) |
| SSBP2 | SSBP2_F2 | ACATGTATACTTTAATGAATGC | SSBP2_R3 | ATGGGACCATCTGACCCAG |
| | | AGTACC (171) | | (172) |
| SSBP2 | SSBP2_F1 | GCCACCAGGAACACCCAT | SSBP2_R7 | TCCAGGAGGTACTGCATTCA |
| | | (173) | | TT (174) |
| SSBP2 | SSBP2_F1 | GCCACCAGGAACACCCAT | SSBP2_R8 | TAGGTCCAGGAGGTACTGCA |
| | | (175) | | (176) |
| PDK3 | PDK3_F1 | AACAGTATTACCTGGTAGCTCC | PDK3_R1 | AGGGCACATAAACCACCTGA |
| | | (177) | | (178) |
| PDK3 | PDK3_F2 | ATCGATCCCACCTGTAACGT | PDK3_R2 | CAGGTAATACTGTTCACACA |
| | | (179) | | GCA (180) |
| PDK3 | PDK3_F3 | GAACAGTATTACCTGGTAGCTCC | PDK3_R3 | GAGGGCACATAAACCACCTG |
| | | (181) | | (182) |
| PDK3 | PDK3_F2 | ATCGATCCCACCTGTAACGT | PDK3_R4 | CCAGGTAATACTGTTCACAC |
| | | (183) | | AGC (184) |
| PDK3 | PDK3_F3 | GAACAGTATTACCTGGTAGCTCC | PDK3_R5 | GGGCACATAAACCACCTGAA |
| | | (185) | | (186) |
| PDK3 | PDK3_F1 | AACAGTATTACCTGGTAGCTCC | PDK3_R6 | GTGAGGGCACATAAACCACC |
| | | (187) | | (188) |
| PDK3 | PDK3_F2 | ATCGATCCCACCTGTAACGT | PDK3_R7 | CAGGTAATACTGTTCACACA |
| | | (189) | | GC (190) |
| PDK3 | PDK3_F4 | TCGCCGCTCTCCATCAAA | PDK3_R8 | GCACAGGAAGTTCCTTTCGT |
| | | (191) | | A (192) |
| ZNF318 | ZNF318_FP1 | GAAGATCTCTGATGAGAAGA | ZNF318_RP1 | GCTTCCCGGTCATTCTTTAGT |
| | | ACCG (193) | | T (194) |
| ZNF318 | ZNF318_FP2 | TGCCATAAAGCGCACTGAC | ZNF318_RP2 | GGCAATAAAATCCACTGATG |
| | | (195) | | GGA (196) |
| ZNF318 | ZNF318_FP3 | GTACTATCTTAGGACCGAGTT | ZNF318_RP3 | GCCATCCTTCTCCCTTCGTT |
| | | AGAG (197) | | (198) |
| ZNF318 | ZNF318_FP4 | ATGCCATAAAGCGCACTGAC | ZNF318_RP4 | GGCAATAAAATCCACTGATG |
| | | (199) | | GG (200) |
| ZNF318 | ZNF318_FP5 | TGTACTATCTTAGGACCGAGT | ZNF318_RP5 | GCCATCCTTCTCCCTTCGTT |
| | | TAGAG (201) | | (202) |
| ZNF318 | ZNF318_FP6 | AGATGTACTATCTTAGGACCG | ZNF318_RP6 | CCATCCTTCTCCCTTCGTTT |
| | | AGT (203) | | (204) |
| ZNF318 | ZNF318_FP7 | GCCATAAAGCGCACTGACAA | ZNF318_RP4 | GGCAATAAAATCCACTGATG |
| | | (205) | | GG (206) |
| ZNF318 | ZNF318_FP8 | GTACTATCTTAGGACCGAGTT | ZNF318_RP7 | CCATCCTTCTCCCTTCGTTTC |
| | | AGAG (207) | | (208) |
| HMGN1 | HMGN1_FP1 | AAGACTTACCTGCGGAAAACG | HMGN1_RP1 | TGGCTTCTTTCTCTCCTGCT |
| | | (209) | | (210) |
| HMGN1 | HMGN1_FP2 | AGACTTACCTGCGGAAAACG | HMGN1_RP2 | TTGGCTTCTTTCTCTCCTGC |
| | | (211) | | (212) |
| HMGN1 | HMGN1_FP1 | AAGACTTACCTGCGGAAAACG | HMGN1_RP3 | CTTGGCTTCTTTCTCTCCTGC |
| | | (213) | | (214) |
| HMGN1 | HMGN1_FP3 | GACTTACCTGCGGAAAACGG | HMGN1_RP2 | TTGGCTTCTTTCTCTCCTGC |
| | | (215) | | (216) |
| HMGN1 | HMGN1_FP2 | AGACTTACCTGCGGAAAACG | HMGN1_RP1 | TGGCTTCTTTCTCTCCTGCT |
| | | (217) | | (218) |
| HMGN1 | HMGN1_FP1 | AAGACTTACCTGCGGAAAACG | HMGN1_RP2 | TTGGCTTCTTTCTCTCCTGC |
| | | (219) | | (220) |
| FOXP1 | FOXPLFP1 | CAACGAGAGTGACAGCAGTC | FOXP1_RP1 | GGCTCTTCTTTGACGTGTACA |
| | | (221) | | (222) |
| FOXP1 | FOXP1_FP1 | CAACGAGAGTGACAGCAGTC | FOXP1_RP2 | GGGCTCTTCTTTGACGTGTA |
| | | (223) | | (224) |
| FOXP1 | FOXP1_FP2 | CGCCTACTGCACACCTCTC | FOXP1_RP3 | CATGGAAGCGGTAGTGTATA |
| | | (225) | | GAG (226) |
| FOXP1 | FOXP1_FP2 | CGCCTACTGCACACCTCTC | FOXP1_RP4 | CCATGGAAGCGGTAGTGTAT |
| | | (227) | | AG (228) |
| FOXP1 | FOXP1_FP2 | CGCCTACTGCACACCTCTC | FOXP1_RP5 | CCATGGAAGCGGTAGTGTA |
| | | (229) | | (230) |
| FOXP1 | FOXP1_FP3 | AGAGCAGCCACGCCTACT | FOXP1_RP3 | CATGGAAGCGGTAGTGTATA |
| | | (231) | | GAG (232) |
| FOXP1 | FOXP1_FP4 | CGAAGGCCACAAAAGATCA | FOXP1_RP6 | GCATTGAGAGGTGTGCAGTA |
| | | (233) | | (234) |
| FOXP1 | FOXP1_FP5 | ATGGACAGTGGATGAAGTAGA | FOXP1_RP7 | GCTGCTCTGCATGTTTTTAAT |
| | | ATTC (235) | | AA (236) |
| KIAA0226L | KIAA0226L_ | TCTTCCCATTTCAGACAGCA | KIAA0226L_ | GAGGACTGGAAGCACTGTTT |
| | F1 | (237) | R1 | (238) |
| KIAA0226L | KIAA0226L_ | TCTTCCCATTTCAGACAGCA | KIAA0226L_ | GGAGGACTGGAAGCACTGT |
| | F1 | (239) | R2 | (240) |
| KIAA0226L | KIAA0226L_ | AGCAAGAGTCTGGGTCTTCTA | KIAA0226L_ | GTTTCAGTCACTGGGCTGAC |
| | F2 | (241) | R3 | (242) |
| KIAA0226L | KIAA0226L_ | AGCAAGAGTCTGGGTCTTCT | KIAA0226L_ | TTTCAGTCACTGGGCTGAC |
| | F3 | (243) | R4 | (244) |
| KIAA0226L | KIAA0226L_ | ACAGAAACCTGTAGCTG | KIAA0226L_ | GCTCTTTGGCTAATAGTTCTG |
| | F4 | TTCC (245) | R5 | CA (246) |
| KIAA0226L | KIAA0226L_ | ACAGAAACCTGTAGCTG | KIAA0226L_ | GCTCTTTGGCTAATAGTTCTG |
| | F4 | TTCC (247) | R6 | C (248) |
| KIAA0226L | KIAA0226L_ | AATTCTCAGCTGGCAGG | KIAA0226L_ | GATTCAAAGTCTTTTCGGAC |
| | F5 | TTC (249) | R7 | ACA (250) |
| KIAA0226L | KIAA0226L_ | TGGGCTCCTCCTAGATT | KIAA0226L_ | AGAAAAAATTCTGGGCTGCC |
| | F6 | TCA (251) | R8 | A (252) |

TABLE 4
Probe sequences

| Gene | Probe_label | 5′ to 3′ Sequence (SEQ ID NO) |
|---|---|---|
| PTK2 | CS_PTK2_JA270_5B | CAAGGGCTGCAATCCCACACATCTTGC (253) |
| | CS_PTK2_JA270_1C | AAGTCTTCAGGGTCCGATTGGAAACCAACA (254) |
| | CS_PTK2_JA270_2C | AGGCATTTATATGAGTCCAGAGAATCCAGCTTTGG (255) |
| | CS_PTK2_JA270_4B | AGGTGCACCCGAGCCTCTGACAG (256) |
| | CS_PTK2_JA270_3A | CAAAAGATTTGTACACAGGGACATTGCTGCTCG (257) |
| | CS_PTK2_JA270_7 | TAACGGACAAGGGCTGCAATCCCACAC (258) |
| LRMP | LRMP_JA270_1 | TGACCCAAGTATGGAAGAGAATGGTGTTGAACG (259) |
| | LRMP_JA270_2 | AGAGGCCCAAGGCACAAGTCCAG (260) |
| | LRMP_JA270_3 | ACATGCTTCAGGAGACTCTGTGGTTTCCC (261) |
| | LRMP_JA270_4 | AGCCATCAATCAGGAAAGCCGGGTTAGTA (262) |
| | LRMP_JA270_5 | GCTTCTCTAAACTCCAAGCCATCTTCTCTACGAAGAG |
| | LRMP_JA270_6 | (263) |
| | LRMP_JA270_7 | GTGGGATGTCTCTTCAGTTTATGACACAATAGCTTCC |
| | | (264) |
| | | ACTATTAGAGTCTTTAACACCTCTGTGTGAAGATGACA |
| | | (265) |
| BCL6 | BCL6_HEX1 | AGGAGAGAAACCTTACCATTGTGAGAAGTGTAACCT |
| | BCL6_HEX2 | (266) |
| | BCL6_HEX3 | GGAAGTTTATTAAGGCCAGTGAAGCAGAGATGGTTT |
| | | (267) |
| | | AATAACATCGTTAACAGGTCCATGACGGGCTC (268) |
| PIM2 | PIM2_HEX1 | ACAGATCGACTCCAGGTGGCCATCAAAG (269) |
| | PIM2_HEX2 | GCTGGTCCCCCTTGTCAGACTCAGT (270) |
| | PIM2_HEX3 | CTACACTGACTTTGATGGGACAAGGGTGTACA (271) |
| | PIM2_HEX7 | TCACATGCCCACTCGAAGTCGCA (272) |
| | PIM2_HEX8 | CTGACTTTGATGGGACAAGGGTGTACA (273) |
| | PIM2_HEX9 | CTGGTGCCCTGCTTCATGATGAACC (274) |
| | PIM2_HEX10 | CACTGACTTTGATGGGACAAGGGTG (275) |
| ARID3A | ARID3A_FAM1 | AGCAGTTTAAGCAGCTCTACGAACTCGACG (276) |
| | ARID3A_FAM2 | TGCAGAAGCGAGGGACACCTGTGA (277) |
| | ARID3A_FAM3 | CCCTGCGGACCCAATACATGAAGTACCT (278) |
| | ARID3A_FAM6 | CAACAGCCAAGCCTCCGAAAGCCG (279) |
| | ARID3A_FAM7 | CGGCATCATGTACACAGGAGTTCTGTTTGCTCA (280) |
| CCND2 | CCND2_HEX1 | AGTTTGCCATGTACCCACCGTCGA (281) |
| | CCND2_HEX2 | CTTTAAGTTTGCCATGTACCCACCGTCG (282) |
| | CCND2_HEX3 | TTGCCATGTACCCACCGTCGATGAT (283) |
| FAM46C | FAM46C_JA270_1 | TTCTATTGCCCAGTTTCCCCAGCCAGAA (284) |
| | FAM46C_JA270_2 | CTCTTCTATTGCCCAGTTTCCCCAGCCAG (285) |
| | FAM46C_JA270_3 | CTCCTCTTCTATTGCCCAGTTTCCCCAGC (286) |
| TCF4 | TCF4_FAM1 | TCCTTCTTCATGCAAGATGGCCATCACAGC (287) |
| | TCF4_FAM2 | AGGACCCTTACAGAGGCATGCCACC (288) |
| | TCF4_FAM3 | TGGAGCAGCAAGTCCGAGAAAGGAATCTGAA (289) |
| JADE3 | JADE3_FAM1 | AGAAACCTGCTGAGGTATTCCGGAAGGAC (290) |
| | JADE3_FAM2 | CACAGCCTTCTCTCAGGATTATAGCTGAGAAGGT (291) |
| | JADE3_FAM3 | CTTGCAGAAATGGGTTGTGGGCCAGTT (292) |
| | JADE3_FAM5 | TGTGTGCATCAGGCCTGCTATGGC (293) |
| | JADE3_FAM6 | TGTGGATCCCAGAGGTCAGCATTGCTTG (294) |
| | JADE3_FAM7 | AGGACCTGGAGAGGGTCCGAAATCTGT (295) |
| | JADE3_FAM8 | CCAAGAAATTGATGCAGGGCTTCCTTTGACAAATG (296) |
| SSBP2 | SSBP2_FAM1 | CATGCCTAGTCCAGCAGATTCAACCAACTCT (297) |
| | SSBP2_FAM2 | CCTGGACCTAACAGACCTAATTTTCCAATGGG (298) |
| PDK3 | PDK3_FAM1 | TTGAAGAATTCAATGCCAAAGCGCCAGACAAA (299) |
| | PDK3_FAM2 | CGGATGTGGTGAAAGATGCATATGAAACAGCC (300) |
| | PDK3_FAM3 | CAATTCCTGGACTTCGGGAGAGATAATGCATGTG (301) |
| ZNF318 | ZNF318_HEX1 | TGCTTCCCAGAAGCAAAAGGTTATTGAAGAGAGG (302) |
| | ZNF318_HEX2 | TAACTGTTCCTGCAAAAGGCTCTGAGTTTCTGG (303) |
| | ZNF318_HEX3 | CGGCTTCATAAACAACAAGGAGAAATGCTGCGC (304) |
| HMGN1 | HMGN1_HEX1 | CGAAGACTGAGGAGAGTCCAGCCTCT (305) |
| | HMGN1_HEX2 | AACGAAGACTGAGGAGAGTCCAGCCTC (306) |
| | HMGN1_HEX3 | AAGACTGAGGAGAGTCCAGCCTCTGATG (307) |
| FOXP1 | FOXP1_JA270-1 | CCACAAAAGATCAGTGGTAACCCTTCCC (308) |
| | FOXP1_JA270_2 | TCCTATGCAAGCCGTGCATCC (309) |
| KIAA0226L | KIAA0226L_JA270_1 | TGTAGAAGATGTTCAGCGTGCAGGGCTT (310) |
| | KIAA0226L_JA270_2 | CATACAGTGGCTATGAAGGTTGTGCTGTGTTACA (311) |
| | KIAA0226L_JA270_3 | TGCAGCTCCTCTAAGAGTGTCACTTATGAGCC (312) |
| | KIAA0226L_JA270_4 | TGCAGCTGGCTCGATAGTCGTAAATGAAGA (313) |
| | KIAA0226L_JA270_5 | TTCATCCACCACTCAAGAGGGACCTTGTG (314) |

### Validation of the 15-Gene Signature

The qRT-PCR classifier was validated in commercially acquired DLBCL FFPET specimens (validation cohort 2; n=29, and validation cohort 3; n=46). Concordance rate between qRT-PCR and Affymetrix microarray-based classifiers was 97.1% (Tables 5 and 6).

TABLE 5
Validation of qRT-PCR COO subtype classifier (Cohort 2, n = 29)

| | qRT-PCR COO classifier | |
|---|---|---|
| | ABC | GCB |
| Affymetrix | | |
| ABC | 7 | 0 |
| GCB | 0 | 18 |
| Unclassified | 1 | 3 |

TABLE 6
Validation of qRT-PCR COO subtype classifier (Cohort 3, n = 46)

| | qRT-PCR COO classifier | |
|---|---|---|
| | ABC | GCB |
| Affymetrix | | |
| ABC | 8 | 0 |
| GCB | 2 | 34 |
| Unclassified | 0 | 2 |

The high concordance of the DLBCL subtype classification signature in two independent DLBCL cohorts is surprising, especially given the relatively small number of genes in the signature. These results show that the DLBCL classifier can be used for quick-turn around, simple, inexpensive, and accurate determination of COO subtype.

### Validation of the 6-Gene Signature

The qRT-PCR classifier with 6 of the genes was validated in commercially acquired DLBCL FFPET specimens (validation cohort n=50). The genes included in the 6-gene signature includes the ABC genes CCND2, FOXP1, and JADE3, and the GCB genes ZNF318, SSBP2, and PTK2. Concordance rate between qRT-PCR and Affymetrix microarray-based classifiers was 95% (Table 7).

TABLE 7
Validation of qRT-PCR COO subtype classifier (6-gene classifier)

| 6-gene COO | Affymetrix | | | |
|---|---|---|---|---|
| classifier | GCB | ABC | Unclassified | |
| GCB | 28 | 1 | 2 | n = 31 |
| ABC | 1 | 11 | 3 | n = 15 |
| Unclassified | 2 | | 2 | n = 4 |

The high concordance of a small 6-gene DLBCL subtype classification signature is surprising. These results show that the 6-gene DLBCL classifier can be used for quick-turn around, simple, inexpensive, and accurate determination of COO subtype.

1-15. (canceled)

16. A method of determining the cell of origin (COO) subtype for an individual with diffuse large B cell lymphoma (DLBCL) comprising:
(a) obtaining a sample from the individual (DLBCL sample);
(b) detecting by qRT-PCR the expression of germinal center B cell (GCB) markers ZNF318, PDK3, HMGN1, PTK2, SSBP2, BCL6, and LRMP in the DLBCL sample;
(c) detecting by qRT-PCR the expression of activated B cell (ABC) markers ARID3A, CCND2, FOXP1, KIAA0226L, JADE3, PIM2, TCF4, and FAM46C in the DLBCL sample;
(d) detecting by qRT-PCR the expression of a control gene in the DLBCL sample; and
(e) determining that the COO subtype of the individual is
(i) GCB if the ratio of GCB marker expression to ABC marker expression is higher than a GCB threshold value; or
(ii) ABC if the ratio of ABC marker expression to GCB marker expression is higher than an ABC threshold value.

17. The method according to claim 16 comprising steps (a) to (d), wherein (b) and/or (c) are carried out as follows:
(b) detecting by qRT-PCR the expression of germinal center B cell (GCB) markers ZNF318, PTK2, and SSBP2 in the DLBCL sample;
(c) detecting by qRT-PCR the expression of activated B cell (ABC) markers CCND2, FOXP1, and JADE3 in the DLBCL sample.

18. The method of claim 16, wherein the GCB threshold value is set based on the ratio of GCB marker expression to ABC marker expression in a GCB positive control.

19. The method of claim 16, wherein the ABC threshold value is set based on ABC marker expression to GCB marker expression in an ABC positive control.

20. The method of claim 16, wherein the sample is a from a lung biopsy or bronchoalveolar lavage.

21. The method of claim 16, wherein the sample is formalin-fixed paraffin embedded tissue (FFPET).

22. The method of claim 16, wherein the sample is blood, plasma, or serum.

23. The method of claim 16, wherein the detecting of (b) and (c) are carried out in multiplex in multiple vessels.

24. The method of claim 16, wherein each GCB and ABC marker is individually detected.

25. The method of claim 16, wherein the detecting of (b) is carried out in a single vessel for each sample.

26. The method of claim 16, wherein the detecting of (c) is carried out in a single vessel for each sample.

27. The method of claim 16, wherein the detecting of (d) is carried out in the same vessel as the detecting of (b) and (c).

28. The method of claim 16, comprising adjusting the level of expression detected for the genes in steps (b) and (c) based on the expression detected of the control gene in (d).

29-44. (canceled)


SEQUENCE LISTING (314 sequences)

SEQ ID NO: 1 - DNA, 20 bp, Artificial Sequence
  source: /note=“Description of Artificial Sequence Synthetic primer”
ggcagtattgacagggagga

SEQ ID NO: 2 - DNA, 19 bp, Artificial Sequence
  source: /note=“Description of Artificial Sequence Synthetic primer”
tggtttacccacaggctga

SEQ ID NO: 3 - DNA, 21 bp, Artificial Sequence
  source: /note=“Description of Artificial Sequence Synthetic primer”
ggagaaggccaatttggagat

SEQ ID NO: 4 - DNA, 28 bp, Artificial Sequence
  source: /note=“Description of Artificial Sequence Synthetic primer”
acagtttttacatgttttaattgcaacc

SEQ ID NO: 5 - DNA, 25 bp, Artificial Sequence
  source: /note=“Description of Artificial Sequence Synthetic primer”
ggacagaaaaggaatgctacaacta

SEQ ID NO: 6 - DNA, 20 bp, Artificial Sequence
  source: /note=“Description of Artificial Sequence Synthetic primer”
cgcaatggttagggatggtg

SEQ ID NO: 7 - DNA, 21 bp, Artificial Sequence
  source: /note=“Description of Artificial Sequence Synthetic primer”
agcttagtacagctcttgcat

SEQ ID NO: 8 - DNA, 23 bp, Artificial Sequence
  source: /note=“Description of Artificial Sequence Synthetic primer”
tcatttgaggacaccagaacatt

SEQ ID NO: 9 - DNA, 21 bp, Artificial Sequence
  source: /note=“Description of Artificial Sequence Synthetic primer”
gcccagaagaaggaatcagtt

SEQ ID NO: 10 - DNA, 21 bp, Artificial Sequence
  source: /note=“Description of Artificial Sequence Synthetic primer”
ggtttgcacttgagtgaagtc

SEQ ID NO: 11 - DNA, 19 bp, Artificial Sequence
  source: /note=“Description of Artificial Sequence Synthetic primer”
accattcccctcctaccag

SEQ ID NO: 12 - DNA, 20 bp, Artificial Sequence
  source: /note=“Description of Artificial Sequence Synthetic primer”
ctcacccaggtcagagttca

SEQ ID NO: 13 - DNA, 21 bp, Artificial Sequence
  source: /note=“Description of Artificial Sequence Synthetic primer”
gacctcaggagatagcaatgt

SEQ ID NO: 14 - DNA, 18 bp, Artificial Sequence
  source: /note=“Description of Artificial Sequence Synthetic primer”
cacttgcccaatccctcg

SEQ ID NO: 15 - DNA, 20 bp, Artificial Sequence
  source: /note=“Description of Artificial Sequence Synthetic primer”
gaatggaacctcgcagtcat

SEQ ID NO: 16 - DNA, 21 bp, Artificial Sequence
  source: /note=“Description of Artificial Sequence Synthetic primer”
ggccaactttggtattgatgg

SEQ ID NO: 17 - DNA, 20 bp, Artificial Sequence
  source: /note=“Description of Artificial Sequence Synthetic primer”
ttcgacgttttacctcagct

SEQ ID NO: 18 - DNA, 20 bp, Artificial Sequence
  source: /note=“Description of Artificial Sequence Synthetic primer”
ggcttcacaccatgcatcag

SEQ ID NO: 19 - DNA, 20 bp, Artificial Sequence
  source: /note=“Description of Artificial Sequence Synthetic primer”
gaaagaaggtgaacgggctt

SEQ ID NO: 20 - DNA, 18 bp, Artificial Sequence
  source: /note=“Description of Artificial Sequence Synthetic primer”
gtgtgtccgcatgccttg

SEQ ID NO: 21 - DNA, 22 bp, Artificial Sequence
  source: /note=“Description of Artificial Sequence Synthetic primer”
cctgtctggataatcatggagc

SEQ ID NO: 22 - DNA, 26 bp, Artificial Sequence
  source: /note=“Description of Artificial Sequence Synthetic primer”
gctagatccaaactgtatttccttac

SEQ ID NO: 23 - DNA, 19 bp, Artificial Sequence
  source: /note=“Description of Artificial Sequence Synthetic primer”
cggcccaggtttactgaac

SEQ ID NO: 24 - DNA, 21 bp, Artificial Sequence
  source: /note=“Description of Artificial Sequence Synthetic primer”
tcttcttgctgagccttctct

SEQ ID NO: 25 - DNA, 20 bp, Artificial Sequence
  source: /note=“Description of Artificial Sequence Synthetic primer”
caggctgcatcaggatgaat

SEQ ID NO: 26 - DNA, 18 bp, Artificial Sequence
  source: /note=“Description of Artificial Sequence Synthetic primer”
cagcaggctctcaggaca

SEQ ID NO: 27 - DNA, 19 bp, Artificial Sequence
  source: /note=“Description of Artificial Sequence Synthetic primer”
ctctcaggctgcatcagga

SEQ ID NO: 28 - DNA, 18 bp, Artificial Sequence
  source: /note=“Description of Artificial Sequence Synthetic primer”
cagcaggctctcaggaca

SEQ ID NO: 29 - DNA, 20 bp, Artificial Sequence
  source: /note=“Description of Artificial Sequence Synthetic primer”
acaagatcagcttctcccac

SEQ ID NO: 30 - DNA, 21 bp, Artificial Sequence
  source: /note=“Description of Artificial Sequence Synthetic primer”
ctcgtagagtcttggaatgca

SEQ ID NO: 31 - DNA, 20 bp, Artificial Sequence
  source: /note=“Description of Artificial Sequence Synthetic primer”
caagatcagcttctcccacg

SEQ ID NO: 32 - DNA, 23 bp, Artificial Sequence
  source: /note=“Description of Artificial Sequence Synthetic primer”
ctcgtagagtcttggaatgcaat

SEQ ID NO: 33 - DNA, 25 bp, Artificial Sequence
  source: /note=“Description of Artificial Sequence Synthetic primer”
aggaaccagaaacaatagaagaaca

SEQ ID NO: 34 - DNA, 22 bp, Artificial Sequence
  source: /note=“Description of Artificial Sequence Synthetic primer”
cgatttcacagtggttacagga

SEQ ID NO: 35 - DNA, 21 bp, Artificial Sequence
  source: /note=“Description of Artificial Sequence Synthetic primer”
gccaaagaggaaccagaaaca

SEQ ID NO: 36 - DNA, 21 bp, Artificial Sequence
  source: /note=“Description of Artificial Sequence Synthetic primer”
accgatttcacagtggttaca

SEQ ID NO: 37 - DNA, 20 bp, Artificial Sequence
  source: /note=“Description of Artificial Sequence Synthetic primer”
cagtagggctgagatgttgg

SEQ ID NO: 38 - DNA, 22 bp, Artificial Sequence
  source: /note=“Description of Artificial Sequence Synthetic primer”
acgtgctgaatcatcacttcaa

SEQ ID NO: 39 - DNA, 18 bp, Artificial Sequence
  source: /note=“Description of Artificial Sequence Synthetic primer”
ggccagtagggctgagat

SEQ ID NO: 40 - DNA, 22 bp, Artificial Sequence
  source: /note=“Description of Artificial Sequence Synthetic primer”
acgtgctgaatcatcacttcaa

SEQ ID NO: 41 - DNA, 24 bp, Artificial Sequence
  source: /note=“Description of Artificial Sequence Synthetic primer”
gactgccaaattaaaaaacgttca

SEQ ID NO: 42 - DNA, 24 bp, Artificial Sequence
  source: /note=“Description of Artificial Sequence Synthetic primer”
tttctgggtaaagaggcaatagtc

SEQ ID NO: 43 - DNA, 23 bp, Artificial Sequence
  source: /note=“Description of Artificial Sequence Synthetic primer”
ggaagccaagtctttctgaaaag

SEQ ID NO: 44 - DNA, 21 bp, Artificial Sequence
  source: /note=“Description of Artificial Sequence Synthetic primer”
gaggacttgagatttgttgcc

SEQ ID NO: 45 - DNA, 26 bp, Artificial Sequence
  source: /note=“Description of Artificial Sequence Synthetic primer”
ggcagaagaaaatttgaagaaagaaa

SEQ ID NO: 46 - DNA, 20 bp, Artificial Sequence
  source: /note=“Description of Artificial Sequence Synthetic primer”
aatgatttcctgtgcctggt

SEQ ID NO: 47 - DNA, 21 bp, Artificial Sequence
  source: /note=“Description of Artificial Sequence Synthetic primer”
ggtggctgggatggaaaataa

SEQ ID NO: 48 - DNA, 19 bp, Artificial Sequence
  source: /note=“Description of Artificial Sequence Synthetic primer”
ggacggtgttcactctgct

SEQ ID NO: 49 - DNA, 19 bp, Artificial Sequence
  source: /note=“Description of Artificial Sequence Synthetic primer”
gaagagccacctgcgaatc

SEQ ID NO: 50 - DNA, 20 bp, Artificial Sequence
  source: /note=“Description of Artificial Sequence Synthetic primer”
gctggcttttgtgacggaaa

SEQ ID NO: 51 - DNA, 18 bp, Artificial Sequence
  source: /note=“Description of Artificial Sequence Synthetic primer”
ccacctgcgaatccacac

SEQ ID NO: 52 - DNA, 21 bp, Artificial Sequence
  source: /note=“Description of Artificial Sequence Synthetic primer”
ctggcttttgtgacggaaatg

SEQ ID NO: 53 - DNA, 19 bp, Artificial Sequence
  source: /note=“Description of Artificial Sequence Synthetic primer”
gaagagccacctgcgaatc

SEQ ID NO: 54 - DNA, 19 bp, Artificial Sequence
  source: /note=“Description of Artificial Sequence Synthetic primer”
gctggcttttgtgacggaa

SEQ ID NO: 55 - DNA, 18 bp, Artificial Sequence
  source: /note=“Description of Artificial Sequence Synthetic primer”
ccacctgcgaatccacac

SEQ ID NO: 56 - DNA, 20 bp, Artificial Sequence
  source: /note=“Description of Artificial Sequence Synthetic primer”
gctggcttttgtgacggaaa

SEQ ID NO: 57 - DNA, 19 bp, Artificial Sequence
  source: /note=“Description of Artificial Sequence Synthetic primer”
catgttgtggacacttgcc

SEQ ID NO: 58 - DNA, 20 bp, Artificial Sequence
  source: /note=“Description of Artificial Sequence Synthetic primer”
cttcacgaggaggcttgatg

SEQ ID NO: 59 - DNA, 20 bp, Artificial Sequence
  source: /note=“Description of Artificial Sequence Synthetic primer”
ggagcatgttgtggacactt

SEQ ID NO: 60 - DNA, 20 bp, Artificial Sequence
  source: /note=“Description of Artificial Sequence Synthetic primer”
ctcttcacgaggaggcttga

SEQ ID NO: 61 - DNA, 20 bp, Artificial Sequence
  source: /note=“Description of Artificial Sequence Synthetic primer”
atggagcatgttgtggacac

SEQ ID NO: 62 - DNA, 18 bp, Artificial Sequence
  source: /note=“Description of Artificial Sequence Synthetic primer”
cgaggaggcttgatggca

SEQ ID NO: 63 - DNA, 19 bp, Artificial Sequence
  source: /note=“Description of Artificial Sequence Synthetic primer”
ggactccaccatcccacaa

SEQ ID NO: 64 - DNA, 20 bp, Artificial Sequence
  source: /note=“Description of Artificial Sequence Synthetic primer”
tagagtggtgagtggctctc

SEQ ID NO: 65 - DNA, 18 bp, Artificial Sequence
  source: /note=“Description of Artificial Sequence Synthetic primer”
gtgccctgcttcatgatg

SEQ ID NO: 66 - DNA, 21 bp, Artificial Sequence
  source: /note=“Description of Artificial Sequence Synthetic primer”
ctggtgtcgagagatccactc

SEQ ID NO: 67 - DNA, 18 bp, Artificial Sequence
  source: /note=“Description of Artificial Sequence Synthetic primer”
ggctgtgccaaactcatt

SEQ ID NO: 68 - DNA, 17 bp, Artificial Sequence
  source: /note=“Description of Artificial Sequence Synthetic primer”
gggctgtacacccttgt

SEQ ID NO: 69 - DNA, 18 bp, Artificial Sequence
  source: /note=“Description of Artificial Sequence Synthetic primer”
gtgccctgcttcatgatg

SEQ ID NO: 70 - DNA, 21 bp, Artificial Sequence
  source: /note=“Description of Artificial Sequence Synthetic primer”
catggtactggtgtcgagaga

SEQ ID NO: 71 - DNA, 18 bp, Artificial Sequence
  source: /note=“Description of Artificial Sequence Synthetic primer”
gtgccctgcttcatgatg

SEQ ID NO: 72 - DNA, 18 bp, Artificial Sequence
  source: /note=“Description of Artificial Sequence Synthetic primer”
ccgggagtgcatggtact

SEQ ID NO: 73 - DNA, 18 bp, Artificial Sequence
  source: /note=“Description of Artificial Sequence Synthetic primer”
ggacaccgcctcacagat

SEQ ID NO: 74 - DNA, 20 bp, Artificial Sequence
  source: /note=“Description of Artificial Sequence Synthetic primer”
agtgggcatgtgactgagtc

SEQ ID NO: 75 - DNA, 18 bp, Artificial Sequence
  source: /note=“Description of Artificial Sequence Synthetic primer”
ggacaccgcctcacagat

SEQ ID NO: 76 - DNA, 18 bp, Artificial Sequence
  source: /note=“Description of Artificial Sequence Synthetic primer”
cttcgagtgggcatgtga

SEQ ID NO: 77 - DNA, 18 bp, Artificial Sequence
  source: /note=“Description of Artificial Sequence Synthetic primer”
cgcctcacagatcgactc

SEQ ID NO: 78 - DNA, 18 bp, Artificial Sequence
  source: /note=“Description of Artificial Sequence Synthetic primer”
gcagtgcgacttcgagtg

SEQ ID NO: 79 - DNA, 18 bp, Artificial Sequence
  source: /note=“Description of Artificial Sequence Synthetic primer”
accgtcttcgcaggacac

SEQ ID NO: 80 - DNA, 19 bp, Artificial Sequence
  source: /note=“Description of Artificial Sequence Synthetic primer”
gggcatgtgactgagtctg

SEQ ID NO: 81 - DNA, 19 bp, Artificial Sequence
  source: /note=“Description of Artificial Sequence Synthetic primer”
ggcgactggacttacgagg

SEQ ID NO: 82 - DNA, 21 bp, Artificial Sequence
  source: /note=“Description of Artificial Sequence Synthetic primer”
ccaggaattccttcctcttgg

SEQ ID NO: 83 - DNA, 20 bp, Artificial Sequence
  source: /note=“Description of Artificial Sequence Synthetic primer”
tcctggatgacttgttcagc

SEQ ID NO: 84 - DNA, 20 bp, Artificial Sequence
  source: /note=“Description of Artificial Sequence Synthetic primer”
aggacctgtttggccatgat

SEQ ID NO: 85 - DNA, 18 bp, Artificial Sequence
  source: /note=“Description of Artificial Sequence Synthetic primer”
acgtccatcaccagtgca

SEQ ID NO: 86 - DNA, 20 bp, Artificial Sequence
  source: /note=“Description of Artificial Sequence Synthetic primer”
cttctcacactcgtaggggt

SEQ ID NO: 87 - DNA, 18 bp, Artificial Sequence
  source: /note=“Description of Artificial Sequence Synthetic primer”
acgtccatcaccagtgca

SEQ ID NO: 88 - DNA, 19 bp, Artificial Sequence
  source: /note=“Description of Artificial Sequence Synthetic primer”
cccgcttctcacactcgta

SEQ ID NO: 89 - DNA, 19 bp, Artificial Sequence
  source: /note=“Description of Artificial Sequence Synthetic primer”
cagctgcccatgagcattc

SEQ ID NO: 90 - DNA, 20 bp, Artificial Sequence
  source: /note=“Description of Artificial Sequence Synthetic primer”
tcaggttcacagcagagtcc

SEQ ID NO: 91 - DNA, 19 bp, Artificial Sequence
  source: /note=“Description of Artificial Sequence Synthetic primer”
cagctgcccatgagcattc

SEQ ID NO: 92 - DNA, 19 bp, Artificial Sequence
  source: /note=“Description of Artificial Sequence Synthetic primer”
cgtcaggttcacagcagag

SEQ ID NO: 93 - DNA, 20 bp, Artificial Sequence
  source: /note=“Description of Artificial Sequence Synthetic primer”
agcatgtcggtggagatcaa

SEQ ID NO: 94 - DNA, 18 bp, Artificial Sequence
  source: /note=“Description of Artificial Sequence Synthetic primer”
ttgggagcagaggttggc

SEQ ID NO: 95 - DNA, 20 bp, Artificial Sequence
  source: /note=“Description of Artificial Sequence Synthetic primer”
atcagcatgtcggtggagat

SEQ ID NO: 96 - DNA, 20 bp, Artificial Sequence
  source: /note=“Description of Artificial Sequence Synthetic primer”
tttgttgggagcagaggttg

SEQ ID NO: 97 - DNA, 21 bp, Artificial Sequence
  source: /note=“Description of Artificial Sequence Synthetic primer”
aggacatccaaccctacatgc

SEQ ID NO: 98 - DNA, 20 bp, Artificial Sequence
  source: /note=“Description of Artificial Sequence Synthetic primer”
gaagacctcttcttcgcact

SEQ ID NO: 99 - DNA, 20 bp, Artificial Sequence
  source: /note=“Description of Artificial Sequence Synthetic primer”
cttcattgctctgtgtgcca

SEQ ID NO: 100 - DNA, 19 bp, Artificial Sequence
  source: /note=“Description of Artificial Sequence Synthetic primer”
tgctcccacacttccagtt

SEQ ID NO: 101 - DNA, 20 bp, Artificial Sequence
  source: /note=“Description of Artificial Sequence Synthetic primer”
cttcattgctctgtgtgcca

SEQ ID NO: 102 - DNA, 20 bp, Artificial Sequence
  source: /note=“Description of Artificial Sequence Synthetic primer”
ccacacttccagttgcgatc

SEQ ID NO: 103 - DNA, 20 bp, Artificial Sequence
  source: /note=“Description of Artificial Sequence Synthetic primer”
gactgagctgctggctaaga

SEQ ID NO: 104 - DNA, 18 bp, Artificial Sequence
  source: /note=“Description of Artificial Sequence Synthetic primer”
gagcaccgcctcaatctg

SEQ ID NO: 105 - DNA, 20 bp, Artificial Sequence
  source: /note=“Description of Artificial Sequence Synthetic primer”
ggacatccaaccctacatgc

SEQ ID NO: 106 - DNA, 21 bp, Artificial Sequence
  source: /note=“Description of Artificial Sequence Synthetic primer”
agagggaagacctcttcttcg

SEQ ID NO: 107 - DNA, 20 bp, Artificial Sequence
  source: /note=“Description of Artificial Sequence Synthetic primer”
ggacatccaaccctacatgc

SEQ ID NO: 108 - DNA, 21 bp, Artificial Sequence
  source: /note=“Description of Artificial Sequence Synthetic primer”
ggaagacctcttcttcgcact

SEQ ID NO: 109 - DNA, 20 bp, Artificial Sequence
  source: /note=“Description of Artificial Sequence Synthetic primer”
cttcattgctctgtgtgcca

SEQ ID NO: 110 - DNA, 19 bp, Artificial Sequence
  source: /note=“Description of Artificial Sequence Synthetic primer”
ctgctcccacacttccagt

SEQ ID NO: 111 - DNA, 20 bp, Artificial Sequence
  source: /note=“Description of Artificial Sequence Synthetic primer”
accttcattgctctgtgtgc

SEQ ID NO: 112 - DNA, 19 bp, Artificial Sequence
  source: /note=“Description of Artificial Sequence Synthetic primer”
gctcccacacttccagttg

SEQ ID NO: 113 - DNA, 18 bp, Artificial Sequence
  source: /note=“Description of Artificial Sequence Synthetic primer”
aaggacctgcctctgtcg

SEQ ID NO: 114 - DNA, 20 bp, Artificial Sequence
  source: /note=“Description of Artificial Sequence Synthetic primer”
tctcctctgccatcttcagg

SEQ ID NO: 115 - DNA, 18 bp, Artificial Sequence
  source: /note=“Description of Artificial Sequence Synthetic primer”
ccaaggacctgcctctgt

SEQ ID NO: 116 - DNA, 20 bp, Artificial Sequence
  source: /note=“Description of Artificial Sequence Synthetic primer”
ctcctctgccatcttcaggg

SEQ ID NO: 117 - DNA, 19 bp, Artificial Sequence
  source: /note=“Description of Artificial Sequence Synthetic primer”
ccaaggacctgcctctgtc

SEQ ID NO: 118 - DNA, 20 bp, Artificial Sequence
  source: /note=“Description of Artificial Sequence Synthetic primer”
tctcctctgccatcttcagg

SEQ ID NO: 119 - DNA, 19 bp, Artificial Sequence
  source: /note=“Description of Artificial Sequence Synthetic primer”
ccaaggacctgcctctgtc

SEQ ID NO: 120 - DNA, 20 bp, Artificial Sequence
  source: /note=“Description of Artificial Sequence Synthetic primer”
ctcctctgccatcttcaggg

SEQ ID NO: 121 - DNA, 19 bp, Artificial Sequence
  source: /note=“Description of Artificial Sequence Synthetic primer”
caaggacctgcctctgtcg

SEQ ID NO: 122 - DNA, 20 bp, Artificial Sequence
  source: /note=“Description of Artificial Sequence Synthetic primer”
tctcctctgccatcttcagg

SEQ ID NO: 123 - DNA, 18 bp, Artificial Sequence
  source: /note=“Description of Artificial Sequence Synthetic primer”
ccaaggacctgcctctgt

SEQ ID NO: 124 - DNA, 20 bp, Artificial Sequence
  source: /note=“Description of Artificial Sequence Synthetic primer”
ctctgccatcttcaggggat

SEQ ID NO: 125 - DNA, 18 bp, Artificial Sequence
  source: /note=“Description of Artificial Sequence Synthetic primer”
aaggacctgcctctgtcg

SEQ ID NO: 126 - DNA, 20 bp, Artificial Sequence
  source: /note=“Description of Artificial Sequence Synthetic primer”
tgctctcctctgccatcttc

SEQ ID NO: 127 - DNA, 19 bp, Artificial Sequence
  source: /note=“Description of Artificial Sequence Synthetic primer”
ccaaggacctgcctctgtc

SEQ ID NO: 128 - DNA, 20 bp, Artificial Sequence
  source: /note=“Description of Artificial Sequence Synthetic primer”
ctctgccatcttcaggggat

SEQ ID NO: 129 - DNA, 20 bp, Artificial Sequence
  source: /note=“Description of Artificial Sequence Synthetic primer”
aaaccagcaaccagcacttt

SEQ ID NO: 130 - DNA, 19 bp, Artificial Sequence
  source: /note=“Description of Artificial Sequence Synthetic primer”
gaggagctccaagggtcac

SEQ ID NO: 131 - DNA, 20 bp, Artificial Sequence
  source: /note=“Description of Artificial Sequence Synthetic primer”
caaccagcactttccctagc

SEQ ID NO: 132 - DNA, 19 bp, Artificial Sequence
  source: /note=“Description of Artificial Sequence Synthetic primer”
ccactggaggagctccaag

SEQ ID NO: 133 - DNA, 19 bp, Artificial Sequence
  source: /note=“Description of Artificial Sequence Synthetic primer”
acttcccctgacctgaacc

SEQ ID NO: 134 - DNA, 20 bp, Artificial Sequence
  source: /note=“Description of Artificial Sequence Synthetic primer”
gagacactctgcccctgtag

SEQ ID NO: 135 - DNA, 19 bp, Artificial Sequence
  source: /note=“Description of Artificial Sequence Synthetic primer”
cgacttcccctgacctgaa

SEQ ID NO: 136 - DNA, 20 bp, Artificial Sequence
  source: /note=“Description of Artificial Sequence Synthetic primer”
gagacactctgcccctgtag

SEQ ID NO: 137 - DNA, 20 bp, Artificial Sequence
  source: /note=“Description of Artificial Sequence Synthetic primer”
gcaaccagcactttccctag

SEQ ID NO: 138 - DNA, 20 bp, Artificial Sequence
  source: /note=“Description of Artificial Sequence Synthetic primer”
attcatcccactggaggagc

SEQ ID NO: 139 - DNA, 18 bp, Artificial Sequence
  source: /note=“Description of Artificial Sequence Synthetic primer”
tggccgtcatcctcagtc

SEQ ID NO: 140 - DNA, 21 bp, Artificial Sequence
  source: /note=“Description of Artificial Sequence Synthetic primer”
cctcccttcttttcagacacg

SEQ ID NO: 141 - DNA, 19 bp, Artificial Sequence
  source: /note=“Description of Artificial Sequence Synthetic primer”
gtggccgtcatcctcagtc

SEQ ID NO: 142 - DNA, 20 bp, Artificial Sequence
  source: /note=“Description of Artificial Sequence Synthetic primer”
tcttttcagacacgcagctt

SEQ ID NO: 143 - DNA, 18 bp, Artificial Sequence
  source: /note=“Description of Artificial Sequence Synthetic primer”
gtggccgtcatcctcagt

SEQ ID NO: 144 - DNA, 20 bp, Artificial Sequence
  source: /note=“Description of Artificial Sequence Synthetic primer”
tcttttcagacacgcagctt

SEQ ID NO: 145 - DNA, 26 bp, Artificial Sequence
  source: /note=“Description of Artificial Sequence Synthetic primer”
tcaagtcaaaaattccaaatgaacac

SEQ ID NO: 146 - DNA, 20 bp, Artificial Sequence
  source: /note=“Description of Artificial Sequence Synthetic primer”
tggaagtttcatggcactga

SEQ ID NO: 147 - DNA, 19 bp, Artificial Sequence
  source: /note=“Description of Artificial Sequence Synthetic primer”
taccagccagtccagacac

SEQ ID NO: 148 - DNA, 21 bp, Artificial Sequence
  source: /note=“Description of Artificial Sequence Synthetic primer”
tcggataaacagaacgtcctt

SEQ ID NO: 149 - DNA, 22 bp, Artificial Sequence
  source: /note=“Description of Artificial Sequence Synthetic primer”
ggcttcaggaactcaatgaaga

SEQ ID NO: 150 - DNA, 23 bp, Artificial Sequence
  source: /note=“Description of Artificial Sequence Synthetic primer”
caggacttctactgtcttttcca

SEQ ID NO: 151 - DNA, 22 bp, Artificial Sequence
  source: /note=“Description of Artificial Sequence Synthetic primer”
ggcttcaggaactcaatgaaga

SEQ ID NO: 152 - DNA, 20 bp, Artificial Sequence
  source: /note=“Description of Artificial Sequence Synthetic primer”
ggcgttccaggacttctact

SEQ ID NO: 153 - DNA, 23 bp, Artificial Sequence
  source: /note=“Description of Artificial Sequence Synthetic primer”
gggaatgatatggtgttctgtga

SEQ ID NO: 154 - DNA, 19 bp, Artificial Sequence
  source: /note=“Description of Artificial Sequence Synthetic primer”
ccttctgggaccttgagga

SEQ ID NO: 155 - DNA, 21 bp, Artificial Sequence
  source: /note=“Description of Artificial Sequence Synthetic primer”
actaaatgggctcatgtcagc

SEQ ID NO: 156 - DNA, 20 bp, Artificial Sequence
  source: /note=“Description of Artificial Sequence Synthetic primer”
atcggttccatcctctcagg

SEQ ID NO: 157 - DNA, 23 bp, Artificial Sequence
  source: /note=“Description of Artificial Sequence Synthetic primer”
tgagaatgtttatgcatctacgc

SEQ ID NO: 158 - DNA, 22 bp, Artificial Sequence
  source: /note=“Description of Artificial Sequence Synthetic primer”
agcttctctcgtctgcttatca

SEQ ID NO: 159 - DNA, 20 bp, Artificial Sequence
  source: /note=“Description of Artificial Sequence Synthetic primer”
ggtttgcaagtccagcttct

SEQ ID NO: 160 - DNA, 22 bp, Artificial Sequence
  source: /note=“Description of Artificial Sequence Synthetic primer”
tggtgggtaaaacagtgagttt

SEQ ID NO: 161 - DNA, 18 bp, Artificial Sequence
  source: /note=“Description of Artificial Sequence Synthetic primer”
gccaccaggaacacccat

SEQ ID NO: 162 - DNA, 20 bp, Artificial Sequence
  source: /note=“Description of Artificial Sequence Synthetic primer”
ggtccaggaggtactgcatt

SEQ ID NO: 163 - DNA, 28 bp, Artificial Sequence
  source: /note=“Description of Artificial Sequence Synthetic primer”
acatgtatactttaatgaatgcagtacc

SEQ ID NO: 164 - DNA, 18 bp, Artificial Sequence
  source: /note=“Description of Artificial Sequence Synthetic primer”
tgggaccatctgacccag

SEQ ID NO: 165 - DNA, 29 bp, Artificial Sequence
  source: /note=“Description of Artificial Sequence Synthetic primer”
aacatgtatactttaatgaatgcagtacc

SEQ ID NO: 166 - DNA, 19 bp, Artificial Sequence
  source: /note=“Description of Artificial Sequence Synthetic primer”
atgggaccatctgacccag

SEQ ID NO: 167 - DNA, 18 bp, Artificial Sequence
  source: /note=“Description of Artificial Sequence Synthetic primer”
gccaccaggaacacccat

SEQ ID NO: 168 - DNA, 21 bp, Artificial Sequence
  source: /note=“Description of Artificial Sequence Synthetic primer”
ccaggaggtactgcattcatt

SEQ ID NO: 169 - DNA, 18 bp, Artificial Sequence
  source: /note=“Description of Artificial Sequence Synthetic primer”
gccaccaggaacacccat

SEQ ID NO: 170 - DNA, 21 bp, Artificial Sequence
  source: /note=“Description of Artificial Sequence Synthetic primer”
gtccaggaggtactgcattca

SEQ ID NO: 171 - DNA, 28 bp, Artificial Sequence
  source: /note=“Description of Artificial Sequence Synthetic primer”
acatgtatactttaatgaatgcagtacc

SEQ ID NO: 172 - DNA, 19 bp, Artificial Sequence
  source: /note=“Description of Artificial Sequence Synthetic primer”
atgggaccatctgacccag

SEQ ID NO: 173 - DNA, 18 bp, Artificial Sequence
  source: /note=“Description of Artificial Sequence Synthetic primer”
gccaccaggaacacccat

SEQ ID NO: 174 - DNA, 22 bp, Artificial Sequence
  source: /note=“Description of Artificial Sequence Synthetic primer”
tccaggaggtactgcattcatt

SEQ ID NO: 175 - DNA, 18 bp, Artificial Sequence
  source: /note=“Description of Artificial Sequence Synthetic primer”
gccaccaggaacacccat

SEQ ID NO: 176 - DNA, 20 bp, Artificial Sequence
  source: /note=“Description of Artificial Sequence Synthetic primer”
taggtccaggaggtactgca

SEQ ID NO: 177 - DNA, 22 bp, Artificial Sequence
  source: /note=“Description of Artificial Sequence Synthetic primer”
aacagtattacctggtagctcc

SEQ ID NO: 178 - DNA, 20 bp, Artificial Sequence
  source: /note=“Description of Artificial Sequence Synthetic primer”
agggcacataaaccacctga

SEQ ID NO: 179 - DNA, 20 bp, Artificial Sequence
  source: /note=“Description of Artificial Sequence Synthetic primer”
atcgatcccacctgtaacgt

SEQ ID NO: 180 - DNA, 23 bp, Artificial Sequence
  source: /note=“Description of Artificial Sequence Synthetic primer”
caggtaatactgttcacacagca

SEQ ID NO: 181 - DNA, 23 bp, Artificial Sequence
  source: /note=“Description of Artificial Sequence Synthetic primer”
gaacagtattacctggtagctcc

SEQ ID NO: 182 - DNA, 20 bp, Artificial Sequence
  source: /note=“Description of Artificial Sequence Synthetic primer”
gagggcacataaaccacctg

SEQ ID NO: 183 - DNA, 20 bp, Artificial Sequence
  source: /note=“Description of Artificial Sequence Synthetic primer”
atcgatcccacctgtaacgt

SEQ ID NO: 184 - DNA, 23 bp, Artificial Sequence
  source: /note=“Description of Artificial Sequence Synthetic primer”
ccaggtaatactgttcacacagc

SEQ ID NO: 185 - DNA, 23 bp, Artificial Sequence
  source: /note=“Description of Artificial Sequence Synthetic primer”
gaacagtattacctggtagctcc

SEQ ID NO: 186 - DNA, 20 bp, Artificial Sequence
  source: /note=“Description of Artificial Sequence Synthetic primer”
gggcacataaaccacctgaa

SEQ ID NO: 187 - DNA, 22 bp, Artificial Sequence
  source: /note=“Description of Artificial Sequence Synthetic primer”
aacagtattacctggtagctcc

SEQ ID NO: 188 - DNA, 20 bp, Artificial Sequence
  source: /note=“Description of Artificial Sequence Synthetic primer”
gtgagggcacataaaccacc

SEQ ID NO: 189 - DNA, 20 bp, Artificial Sequence
  source: /note=“Description of Artificial Sequence Synthetic primer”
atcgatcccacctgtaacgt

SEQ ID NO: 190 - DNA, 22 bp, Artificial Sequence
  source: /note=“Description of Artificial Sequence Synthetic primer”
caggtaatactgttcacacagc

SEQ ID NO: 191 - DNA, 18 bp, Artificial Sequence
  source: /note=“Description of Artificial Sequence Synthetic primer”
tcgccgctctccatcaaa

SEQ ID NO: 192 - DNA, 21 bp, Artificial Sequence
  source: /note=“Description of Artificial Sequence Synthetic primer”
gcacaggaagttcctttcgta

SEQ ID NO: 193 - DNA, 24 bp, Artificial Sequence
  source: /note=“Description of Artificial Sequence Synthetic primer”
gaagatctctgatgagaagaaccg

SEQ ID NO: 194 - DNA, 22 bp, Artificial Sequence
  source: /note=“Description of Artificial Sequence Synthetic primer”
gcttcccggtcattctttagtt

SEQ ID NO: 195 - DNA, 19 bp, Artificial Sequence
  source: /note=“Description of Artificial Sequence Synthetic primer”
tgccataaagcgcactgac

SEQ ID NO: 196 - DNA, 23 bp, Artificial Sequence
  source: /note=“Description of Artificial Sequence Synthetic primer”
ggcaataaaatccactgatggga

SEQ ID NO: 197 - DNA, 25 bp, Artificial Sequence
  source: /note=“Description of Artificial Sequence Synthetic primer”
gtactatcttaggaccgagttagag

SEQ ID NO: 198 - DNA, 20 bp, Artificial Sequence
  source: /note=“Description of Artificial Sequence Synthetic primer”
gccatccttctcccttcgtt

SEQ ID NO: 199 - DNA, 20 bp, Artificial Sequence
  source: /note=“Description of Artificial Sequence Synthetic primer”
atgccataaagcgcactgac

SEQ ID NO: 200 - DNA, 22 bp, Artificial Sequence
  source: /note=“Description of Artificial Sequence Synthetic primer”
ggcaataaaatccactgatggg

SEQ ID NO: 201 - DNA, 26 bp, Artificial Sequence
  source: /note=“Description of Artificial Sequence Synthetic primer”
tgtactatcttaggaccgagttagag

SEQ ID NO: 202 - DNA, 20 bp, Artificial Sequence
  source: /note=“Description of Artificial Sequence Synthetic primer”
gccatccttctcccttcgtt

SEQ ID NO: 203 - DNA, 24 bp, Artificial Sequence
  source: /note=“Description of Artificial Sequence Synthetic primer”
agatgtactatcttaggaccgagt

SEQ ID NO: 204 - DNA, 20 bp, Artificial Sequence
  source: /note=“Description of Artificial Sequence Synthetic primer”
ccatccttctcccttcgttt

SEQ ID NO: 205 - DNA, 20 bp, Artificial Sequence
  source: /note=“Description of Artificial Sequence Synthetic primer”
gccataaagcgcactgacaa

SEQ ID NO: 206 - DNA, 22 bp, Artificial Sequence
  source: /note=“Description of Artificial Sequence Synthetic primer”
ggcaataaaatccactgatggg

SEQ ID NO: 207 - DNA, 25 bp, Artificial Sequence
  source: /note=“Description of Artificial Sequence Synthetic primer”
gtactatcttaggaccgagttagag

SEQ ID NO: 208 - DNA, 21 bp, Artificial Sequence
  source: /note=“Description of Artificial Sequence Synthetic primer”
ccatccttctcccttcgtttc

SEQ ID NO: 209 - DNA, 21 bp, Artificial Sequence
  source: /note=“Description of Artificial Sequence Synthetic primer”
aagacttacctgcggaaaacg

SEQ ID NO: 210 - DNA, 20 bp, Artificial Sequence
  source: /note=“Description of Artificial Sequence Synthetic primer”
tggcttctttctctcctgct

SEQ ID NO: 211 - DNA, 20 bp, Artificial Sequence
  source: /note=“Description of Artificial Sequence Synthetic primer”
agacttacctgcggaaaacg

SEQ ID NO: 212 - DNA, 20 bp, Artificial Sequence
  source: /note=“Description of Artificial Sequence Synthetic primer”
ttggcttctttctctcctgc

SEQ ID NO: 213 - DNA, 21 bp, Artificial Sequence
  source: /note=“Description of Artificial Sequence Synthetic primer”
aagacttacctgcggaaaacg

SEQ ID NO: 214 - DNA, 21 bp, Artificial Sequence
  source: /note=“Description of Artificial Sequence Synthetic primer”
cttggcttctttctctcctgc

SEQ ID NO: 215 - DNA, 20 bp, Artificial Sequence
  source: /note=“Description of Artificial Sequence Synthetic primer”
gacttacctgcggaaaacgg

SEQ ID NO: 216 - DNA, 20 bp, Artificial Sequence
  source: /note=“Description of Artificial Sequence Synthetic primer”
ttggcttctttctctcctgc

SEQ ID NO: 217 - DNA, 20 bp, Artificial Sequence
  source: /note=“Description of Artificial Sequence Synthetic primer”
agacttacctgcggaaaacg

SEQ ID NO: 218 - DNA, 20 bp, Artificial Sequence
  source: /note=“Description of Artificial Sequence Synthetic primer”
tggcttctttctctcctgct

SEQ ID NO: 219 - DNA, 21 bp, Artificial Sequence
  source: /note=“Description of Artificial Sequence Synthetic primer”
aagacttacctgcggaaaacg

SEQ ID NO: 220 - DNA, 20 bp, Artificial Sequence
  source: /note=“Description of Artificial Sequence Synthetic primer”
ttggcttctttctctcctgc

SEQ ID NO: 221 - DNA, 20 bp, Artificial Sequence
  source: /note=“Description of Artificial Sequence Synthetic primer”
caacgagagtgacagcagtc

SEQ ID NO: 222 - DNA, 21 bp, Artificial Sequence
  source: /note=“Description of Artificial Sequence Synthetic primer”
ggctcttctttgacgtgtaca

SEQ ID NO: 223 - DNA, 20 bp, Artificial Sequence
  source: /note=“Description of Artificial Sequence Synthetic primer”
caacgagagtgacagcagtc

SEQ ID NO: 224 - DNA, 20 bp, Artificial Sequence
  source: /note=“Description of Artificial Sequence Synthetic primer”
gggctcttctttgacgtgta

SEQ ID NO: 225 - DNA, 19 bp, Artificial Sequence
  source: /note=“Description of Artificial Sequence Synthetic primer”
cgcctactgcacacctctc

SEQ ID NO: 226 - DNA, 23 bp, Artificial Sequence
  source: /note=“Description of Artificial Sequence Synthetic primer”
catggaagcggtagtgtatagag

SEQ ID NO: 227 - DNA, 19 bp, Artificial Sequence
  source: /note=“Description of Artificial Sequence Synthetic primer”
cgcctactgcacacctctc

SEQ ID NO: 228 - DNA, 22 bp, Artificial Sequence
  source: /note=“Description of Artificial Sequence Synthetic primer”
ccatggaagcggtagtgtatag

SEQ ID NO: 229 - DNA, 19 bp, Artificial Sequence
  source: /note=“Description of Artificial Sequence Synthetic primer”
cgcctactgcacacctctc

SEQ ID NO: 230 - DNA, 19 bp, Artificial Sequence
  source: /note=“Description of Artificial Sequence Synthetic primer”
ccatggaagcggtagtgta

SEQ ID NO: 231 - DNA, 18 bp, Artificial Sequence
  source: /note=“Description of Artificial Sequence Synthetic primer”
agagcagccacgcctact

SEQ ID NO: 232 - DNA, 23 bp, Artificial Sequence
  source: /note=“Description of Artificial Sequence Synthetic primer”
catggaagcggtagtgtatagag

SEQ ID NO: 233 - DNA, 19 bp, Artificial Sequence
  source: /note=“Description of Artificial Sequence Synthetic primer”
cgaaggccacaaaagatca

SEQ ID NO: 234 - DNA, 20 bp, Artificial Sequence
  source: /note=“Description of Artificial Sequence Synthetic primer”
gcattgagaggtgtgcagta

SEQ ID NO: 235 - DNA, 25 bp, Artificial Sequence
  source: /note=“Description of Artificial Sequence Synthetic primer”
atggacagtggatgaagtagaattc

SEQ ID NO: 236 - DNA, 23 bp, Artificial Sequence
  source: /note=“Description of Artificial Sequence Synthetic primer”
gctgctctgcatgtttttaataa

SEQ ID NO: 237 - DNA, 20 bp, Artificial Sequence
  source: /note=“Description of Artificial Sequence Synthetic primer”
tcttcccatttcagacagca

SEQ ID NO: 238 - DNA, 20 bp, Artificial Sequence
  source: /note=“Description of Artificial Sequence Synthetic primer”
gaggactggaagcactgttt

SEQ ID NO: 239 - DNA, 20 bp, Artificial Sequence
  source: /note=“Description of Artificial Sequence Synthetic primer”
tcttcccatttcagacagca

SEQ ID NO: 240 - DNA, 19 bp, Artificial Sequence
  source: /note=“Description of Artificial Sequence Synthetic primer”
ggaggactggaagcactgt

SEQ ID NO: 241 - DNA, 21 bp, Artificial Sequence
  source: /note=“Description of Artificial Sequence Synthetic primer”
agcaagagtctgggtcttcta

SEQ ID NO: 242 - DNA, 20 bp, Artificial Sequence
  source: /note=“Description of Artificial Sequence Synthetic primer”
gtttcagtcactgggctgac

SEQ ID NO: 243 - DNA, 20 bp, Artificial Sequence
  source: /note=“Description of Artificial Sequence Synthetic primer”
agcaagagtctgggtcttct

SEQ ID NO: 244 - DNA, 19 bp, Artificial Sequence
  source: /note=“Description of Artificial Sequence Synthetic primer”
tttcagtcactgggctgac

SEQ ID NO: 245 - DNA, 21 bp, Artificial Sequence
  source: /note=“Description of Artificial Sequence Synthetic primer”
acagaaacctgtagctgttcc

SEQ ID NO: 246 - DNA, 23 bp, Artificial Sequence
  source: /note=“Description of Artificial Sequence Synthetic primer”
gctctttggctaatagttctgca

SEQ ID NO: 247 - DNA, 21 bp, Artificial Sequence
  source: /note=“Description of Artificial Sequence Synthetic primer”
acagaaacctgtagctgttcc

SEQ ID NO: 248 - DNA, 22 bp, Artificial Sequence
  source: /note=“Description of Artificial Sequence Synthetic primer”
gctctttggctaatagttctgc

SEQ ID NO: 249 - DNA, 20 bp, Artificial Sequence
  source: /note=“Description of Artificial Sequence Synthetic primer”
aattctcagctggcaggttc

SEQ ID NO: 250 - DNA, 23 bp, Artificial Sequence
  source: /note=“Description of Artificial Sequence Synthetic primer”
gattcaaagtcttttcggacaca

SEQ ID NO: 251 - DNA, 20 bp, Artificial Sequence
  source: /note=“Description of Artificial Sequence Synthetic primer”
tgggctcctcctagatttca

SEQ ID NO: 252 - DNA, 21 bp, Artificial Sequence
  source: /note=“Description of Artificial Sequence Synthetic primer”
agaaaaaattctgggctgcca

SEQ ID NO: 253 - DNA, 27 bp, Artificial Sequence
  source: /note=“Description of Artificial Sequence Synthetic probe”
caagggctgcaatcccacacatcttgc

SEQ ID NO: 254 - DNA, 30 bp, Artificial Sequence
  source: /note=“Description of Artificial Sequence Synthetic probe”
aagtcttcagggtccgattggaaaccaaca

SEQ ID NO: 255 - DNA, 35 bp, Artificial Sequence
  source: /note=“Description of Artificial Sequence Synthetic probe”
aggcatttatatgagtccagagaatccagctttgg

SEQ ID NO: 256 - DNA, 23 bp, Artificial Sequence
  source: /note=“Description of Artificial Sequence Synthetic probe”
aggtgcacccgagcctctgacag

SEQ ID NO: 257 - DNA, 33 bp, Artificial Sequence
  source: /note=“Description of Artificial Sequence Synthetic probe”
caaaagatttgtacacagggacattgctgctcg

SEQ ID NO: 258 - DNA, 27 bp, Artificial Sequence
  source: /note=“Description of Artificial Sequence Synthetic probe”
taacggacaagggctgcaatcccacac

SEQ ID NO: 259 - DNA, 33 bp, Artificial Sequence
  source: /note=“Description of Artificial Sequence Synthetic probe”
tgacccaagtatggaagagaatggtgttgaacg

SEQ ID NO: 260 - DNA, 23 bp, Artificial Sequence
  source: /note=“Description of Artificial Sequence Synthetic probe”
agaggcccaaggcacaagtccag

SEQ ID NO: 261 - DNA, 29 bp, Artificial Sequence
  source: /note=“Description of Artificial Sequence Synthetic probe”
acatgcttcaggagactctgtggtttccc

SEQ ID NO: 262 - DNA, 29 bp, Artificial Sequence
  source: /note=“Description of Artificial Sequence Synthetic probe”
agccatcaatcaggaaagccgggttagta

SEQ ID NO: 263 - DNA, 37 bp, Artificial Sequence
  source: /note=“Description of Artificial Sequence Synthetic probe”
gcttctctaaactccaagccatcttctctacgaagag

SEQ ID NO: 264 - DNA, 37 bp, Artificial Sequence
  source: /note=“Description of Artificial Sequence Synthetic probe”
gtgggatgtctcttcagtttatgacacaatagcttcc

SEQ ID NO: 265 - DNA, 38 bp, Artificial Sequence
  source: /note=“Description of Artificial Sequence Synthetic probe”
actattagagtctttaacacctctgtgtgaagatgaca

SEQ ID NO: 266 - DNA, 36 bp, Artificial Sequence
  source: /note=“Description of Artificial Sequence Synthetic probe”
aggagagaaaccttaccattgtgagaagtgtaacct

SEQ ID NO: 267 - DNA, 36 bp, Artificial Sequence
  source: /note=“Description of Artificial Sequence Synthetic probe”
ggaagtttattaaggccagtgaagcagagatggttt

SEQ ID NO: 268 - DNA, 32 bp, Artificial Sequence
  source: /note=“Description of Artificial Sequence Synthetic probe”
aataacatcgttaacaggtccatgacgggctc

SEQ ID NO: 269 - DNA, 28 bp, Artificial Sequence
  source: /note=“Description of Artificial Sequence Synthetic probe”
acagatcgactccaggtggccatcaaag

SEQ ID NO: 270 - DNA, 25 bp, Artificial Sequence
  source: /note=“Description of Artificial Sequence Synthetic probe”
gctggtcccccttgtcagactcagt

SEQ ID NO: 271 - DNA, 32 bp, Artificial Sequence
  source: /note=“Description of Artificial Sequence Synthetic probe”
ctacactgactttgatgggacaagggtgtaca

SEQ ID NO: 272 - DNA, 23 bp, Artificial Sequence
  source: /note=“Description of Artificial Sequence Synthetic probe”
tcacatgcccactcgaagtcgca

SEQ ID NO: 273 - DNA, 27 bp, Artificial Sequence
  source: /note=“Description of Artificial Sequence Synthetic probe”
ctgactttgatgggacaagggtgtaca

SEQ ID NO: 274 - DNA, 25 bp, Artificial Sequence
  source: /note=“Description of Artificial Sequence Synthetic probe”
ctggtgccctgcttcatgatgaacc

SEQ ID NO: 275 - DNA, 25 bp, Artificial Sequence
  source: /note=“Description of Artificial Sequence Synthetic probe”
cactgactttgatgggacaagggtg

SEQ ID NO: 276 - DNA, 30 bp, Artificial Sequence
  source: /note=“Description of Artificial Sequence Synthetic probe”
agcagtttaagcagctctacgaactcgacg

SEQ ID NO: 277 - DNA, 24 bp, Artificial Sequence
  source: /note=“Description of Artificial Sequence Synthetic probe”
tgcagaagcgagggacacctgtga

SEQ ID NO: 278 - DNA, 28 bp, Artificial Sequence
  source: /note=“Description of Artificial Sequence Synthetic probe”
ccctgcggacccaatacatgaagtacct

SEQ ID NO: 279 - DNA, 24 bp, Artificial Sequence
  source: /note=“Description of Artificial Sequence Synthetic probe”
caacagccaagcctccgaaagccg

SEQ ID NO: 280 - DNA, 33 bp, Artificial Sequence
  source: /note=“Description of Artificial Sequence Synthetic probe”
cggcatcatgtacacaggagttctgtttgctca

SEQ ID NO: 281 - DNA, 24 bp, Artificial Sequence
  source: /note=“Description of Artificial Sequence Synthetic probe”
agtttgccatgtacccaccgtcga

SEQ ID NO: 282 - DNA, 28 bp, Artificial Sequence
  source: /note=“Description of Artificial Sequence Synthetic probe”
ctttaagtttgccatgtacccaccgtcg

SEQ ID NO: 283 - DNA, 25 bp, Artificial Sequence
  source: /note=“Description of Artificial Sequence Synthetic probe”
ttgccatgtacccaccgtcgatgat

SEQ ID NO: 284 - DNA, 28 bp, Artificial Sequence
  source: /note=“Description of Artificial Sequence Synthetic probe”
ttctattgcccagtttccccagccagaa

SEQ ID NO: 285 - DNA, 29 bp, Artificial Sequence
  source: /note=“Description of Artificial Sequence Synthetic probe”
ctcttctattgcccagtttccccagccag

SEQ ID NO: 286 - DNA, 29 bp, Artificial Sequence
  source: /note=“Description of Artificial Sequence Synthetic probe”
ctcctcttctattgcccagtttccccagc

SEQ ID NO: 287 - DNA, 30 bp, Artificial Sequence
  source: /note=“Description of Artificial Sequence Synthetic probe”
tccttcttcatgcaagatggccatcacagc

SEQ ID NO: 288 - DNA, 25 bp, Artificial Sequence
  source: /note=“Description of Artificial Sequence Synthetic probe”
aggacccttacagaggcatgccacc

SEQ ID NO: 289 - DNA, 31 bp, Artificial Sequence
  source: /note=“Description of Artificial Sequence Synthetic probe”
tggagcagcaagtccgagaaaggaatctgaa

SEQ ID NO: 290 - DNA, 29 bp, Artificial Sequence
  source: /note=“Description of Artificial Sequence Synthetic probe”
agaaacctgctgaggtattccggaaggac

SEQ ID NO: 291 - DNA, 34 bp, Artificial Sequence
  source: /note=“Description of Artificial Sequence Synthetic probe”
cacagccttctctcaggattatagctgagaaggt

SEQ ID NO: 292 - DNA, 27 bp, Artificial Sequence
  source: /note=“Description of Artificial Sequence Synthetic probe”
cttgcagaaatgggttgtgggccagtt

SEQ ID NO: 293 - DNA, 24 bp, Artificial Sequence
  source: /note=“Description of Artificial Sequence Synthetic probe”
tgtgtgcatcaggcctgctatggc

SEQ ID NO: 294 - DNA, 28 bp, Artificial Sequence
  source: /note=“Description of Artificial Sequence Synthetic probe”
tgtggatcccagaggtcagcattgcttg

SEQ ID NO: 295 - DNA, 27 bp, Artificial Sequence
  source: /note=“Description of Artificial Sequence Synthetic probe”
aggacctggagagggtccgaaatctgt

SEQ ID NO: 296 - DNA, 35 bp, Artificial Sequence
  source: /note=“Description of Artificial Sequence Synthetic probe”
ccaagaaattgatgcagggcttcctttgacaaatg

SEQ ID NO: 297 - DNA, 31 bp, Artificial Sequence
  source: /note=“Description of Artificial Sequence Synthetic probe”
catgcctagtccagcagattcaaccaactct

SEQ ID NO: 298 - DNA, 32 bp, Artificial Sequence
  source: /note=“Description of Artificial Sequence Synthetic probe”
cctggacctaacagacctaattttccaatggg

SEQ ID NO: 299 - DNA, 32 bp, Artificial Sequence
  source: /note=“Description of Artificial Sequence Synthetic probe”
ttgaagaattcaatgccaaagcgccagacaaa

SEQ ID NO: 300 - DNA, 32 bp, Artificial Sequence
  source: /note=“Description of Artificial Sequence Synthetic probe”
cggatgtggtgaaagatgcatatgaaacagcc

SEQ ID NO: 301 - DNA, 34 bp, Artificial Sequence
  source: /note=“Description of Artificial Sequence Synthetic probe”
caattcctggacttcgggagagataatgcatgtg

SEQ ID NO: 302 - DNA, 34 bp, Artificial Sequence
  source: /note=“Description of Artificial Sequence Synthetic probe”
tgcttcccagaagcaaaaggttattgaagagagg

SEQ ID NO: 303 - DNA, 33 bp, Artificial Sequence
  source: /note=“Description of Artificial Sequence Synthetic probe”
taactgttcctgcaaaaggctctgagtttctgg

SEQ ID NO: 304 - DNA, 33 bp, Artificial Sequence
  source: /note=“Description of Artificial Sequence Synthetic probe”
cggcttcataaacaacaaggagaaatgctgcgc

SEQ ID NO: 305 - DNA, 26 bp, Artificial Sequence
  source: /note=“Description of Artificial Sequence Synthetic probe”
cgaagactgaggagagtccagcctct

SEQ ID NO: 306 - DNA, 27 bp, Artificial Sequence
  source: /note=“Description of Artificial Sequence Synthetic probe”
aacgaagactgaggagagtccagcctc

SEQ ID NO: 307 - DNA, 28 bp, Artificial Sequence
  source: /note=“Description of Artificial Sequence Synthetic probe”
aagactgaggagagtccagcctctgatg

SEQ ID NO: 308 - DNA, 28 bp, Artificial Sequence
  source: /note=“Description of Artificial Sequence Synthetic probe”
ccacaaaagatcagtggtaacccttccc

SEQ ID NO: 309 - DNA, 21 bp, Artificial Sequence
  source: /note=“Description of Artificial Sequence Synthetic probe”
tcctatgcaagccgtgcatcc

SEQ ID NO: 310 - DNA, 28 bp, Artificial Sequence
  source: /note=“Description of Artificial Sequence Synthetic probe”
tgtagaagatgttcagcgtgcagggctt

SEQ ID NO: 311 - DNA, 34 bp, Artificial Sequence
  source: /note=“Description of Artificial Sequence Synthetic probe”
catacagtggctatgaaggttgtgctgtgttaca

SEQ ID NO: 312 - DNA, 32 bp, Artificial Sequence
  source: /note=“Description of Artificial Sequence Synthetic probe”
tgcagctcctctaagagtgtcacttatgagcc

SEQ ID NO: 313 - DNA, 30 bp, Artificial Sequence
  source: /note=“Description of Artificial Sequence Synthetic probe”
tgcagctggctcgatagtcgtaaatgaaga

SEQ ID NO: 314 - DNA, 29 bp, Artificial Sequence
  source: /note=“Description of Artificial Sequence Synthetic probe”
ttcatccaccactcaagagggaccttgtg